(19) United States
(12) Patent Application Publication
(10) Pub. No.: US 2022/0288549 A1
(43) Pub. Date: Sep. 15, 2022

(54) PROCESS FOR PRODUCING MICROPARTICLES LADEN WITH A VOLATILE ORGANIC ACTIVE

(71) Applicant: BASF SE, Ludwigshafen am Rhein (DE)

(72) Inventors: OSCHMANN Dieter Bernd, Ludwigshafen am Rhein (DE); Dominik GARELLA, Ludwigshafen am Rhein (DE); Miika FRANCK, Ludwigshafen am Rhein (DE); Wolfgang KRAUSE, Lampertheim (DE); Thorsten MOHR, Ludwigshafen am Rhein (DE)

(21) Appl. No.: 17/626,134

(22) PCT Filed: Jul. 10, 2020
(86) PCT No.: PCT/EP2020/069538

§ 371 Date: Jan. 11, 2022

(30) Foreign Application Priority Data
Jul. 12, 2019 (EP) 19186110.3

(51) Int. Cl. B01J 13/12; B01J 13/20

(52) U.S. Cl. CPC B01J 13/12; B01J 13/203

(57) ABSTRACT

The present invention relates to a process for producing microparticles laden with at least one active, wherein the microparticles are formed from a thermoplastic organic, polymeric material and in the unladen state, in their interior, have at least one cavity connected via pores to the surface of the microparticles, wherein (a) a composition of unladen microparticles are impregnated with a liquid comprising the active, whereby laden microparticles are obtained, which contain in the interior cavity the liquid, and (b) subjecting the laden microparticles to thermal treatment by passing a stream of free flowing laden microparticles in a carrier gas through a heated zone at a temperature of at least 20 K, in particular at least 40 K, e.g. 20 to 250 K especially 40 to 200 K above the softening temperature of the thermoplastic organic polymeric material, where the average residence time of the laden microparticles in the heated zone is not more than 60 s, in particular not more than 30 s, especially not more than 20 s or not more than 10 s, e.g. in the range of 0.1 to 60 s, in particular in the range of 0.1 to 30 s, more particularly in the range of 0.5 to 20 s and especially in the range of 1 to 10 s. The present invention further relates to compositions of microparticles filled with at least one active, in particular with at least volatile organic active, which is obtainable by a process of the invention, and to the use thereof, especially in a product selected from perfumes, washing and cleaning compositions, cosmetic compositions, personal care compositions, hygiene articles, foods, food supplements, fragrance dispensers and fragrances. The present invention further relates to products comprising an inventive composition of microparticles filled with at least one active, in particular with at least one volatile organic active, and to the use thereof, especially for controlled release of actives of low molecular weight and specifically for controlled release of aroma chemicals.

The present invention relates to processes for producing microparticles laden with at least one volatile organic active, such as aroma chemicals, especially to a process for sealing microparticles laden with at least one volatile organic active. The invention also relates to compositions of microparticles laden with at least one volatile organic active and to the use thereof.

Microcapsules have various different uses as carriers for active substances, for example for crop protecting agents, pharmaceutical agents, fragrances and aromas, but also for reactive substances or catalysts for industrial applications. They typically comprise a polymer material that envelops the material to be encapsulated. Advantages of a formulation of this kind are in particular:
protection of reactive actives from environmental effects;
safe and practical handling of toxic or unstable actives;
controlled release of actives;
prevention of premature mixing of substances;
the handling of liquid actives as solids.

An overview of the methodology of microencapsulation of actives can be found in H. Mollet, A. Grubenmann, “Formulation Technology”, chapter 6.4 (Microencapsulation), Wiley VCH Verlag GmbH, Weinheim 2001, and the literature cited therein.

There have been a variety of descriptions of porous microparticles that consist of a polymer material in spongelike form and can be laden with active ingredients.

EP 467528 describes porous polymeric carrier particles with average particle sizes up to 250 μm and pores on their surface. The porous polymeric carrier particles are produced by suspension polymerization of styrene and a polyester of maleic anhydride/phthalic anhydride/propylene glycol in the presence of pore-forming substances. The particles are proposed for enzymes, catalysts and bacteria.

EP 1679065 describes microparticles comprising biodegradable block copolymers constructed from poly(ethylene glycol) terephthalate (PEGT) and poly(butylene terephthalate) (PBT) and an active ingredient selected from the group of interferons for the controlled release of the active ingredient.

US 2016/0168349 describes microparticles having a multimodal pore size distribution. The microparticles contain a polymeric material that is formed from a thermoplastic composition, which is strained to a certain degree to achieve a porous network structure. The microparticles may contain an active agent.

EP 2 431 411 describes a dispersed composition formed by using an auxiliary component (B) comprising an oligosaccharide and a water-soluble plasticizing component for plasticizing the oligosaccharide and kneadable with a resin component (A) comprising a thermoplastic resin. The auxiliary component (B) may form a continuous phase in an islands-in-the-sea structure or in a bicontinuous phase. The kneaded composition may be shaped to prepare a preliminary shaped article which may be immersed in a solvent to elute or wash out the auxiliary component to obtain a shaped porous article. The porous material may be utilized for a separation membrane for liquid, a filter, a moisture absorbent, an adsorbent, a humectant, or an image-receiving layer for recording sheet or as an additive for daily commodity (such as a cosmetic preparation).

EP 2 233 557 describes a process for preparing a perfume encapsulate for use in laundry detergent compositions. The process comprises emulsifying a hydrophobic perfume-copolymer complex in water to form an oil in water emulsion; at least partially dissolving an encapsulation material in water, removing at least some water from the oil in water emulsion comprising the hydrophobic perfume-polymer complex and the encapsulating material to form an encapsulate. The encapsulate is said to have good dissolution profiles, whilst retaining good thermal stability profiles.

WO 2011/088229 and US 2019/0183802 describe porous microparticles composed of biodegradable polymers, e.g. poly(lactide-co-glycolide) (PLGA), comprising, in the pores, an ionic species, e.g. an inorganic salt of a polyvalent ion, which is capable of binding to the active. After the loading with the active, which is generally a biologically active polymer, e.g. a protein, a lipoprotein, a proteoglycan or a nucleic acid, the pores are sealed by heating.

WO 2015/070172 describes porous microparticles composed of biodegradable polymers wherein the pores comprise, as active, a biologically active polymer, e.g. a protein, a lipoprotein, a proteoglycan or a nucleic acid, and an ionic biopolymer, especially an ionic polysaccharide, and a pH modifier, e.g. magnesium carbonate or zinc carbonate. The ionic biopolymer forms an ionic complex with the biologically active polymer. After the microparticles have been laden with a biologically active polymer, e.g. a protein, a lipoprotein, a proteoglycan or a nucleic acid, the pores are sealed by heating.

The methods described in the aforementioned prior art documents teach the loading of porous microparticles with biologically active polymers that are to be released rapidly at the site of use. The continuous release of a volatile organic active over a prolonged period of time is of no significance here. Indeed, such a release is not wanted. Moreover, biologically active polymers are very hydrophilic. There is no description of the loading of the porous microparticles with volatile organic substances or even hydrophobic organic substances such as aroma chemicals. In all processes, the laden microparticles, after the loading, are heated for a prolonged period of generally several hours up to several days in order to close the pores and to prevent premature exit of the active. This regularly leads to stress on the active ingredient and can lead to unwanted degradation of the active and may cause agglomeration or even complete destruction of the laden microparticles.

WO 2018/065481 describes a process for filling porous microparticles with an aroma chemical by suspending the microparticles in a liquid aroma chemical or solution of the aroma chemical. Here too, the sealing is effected by heating over a prolonged period, which can lead to degradation of the active and to unwanted agglomeration or even destruction of the laden microparticles. Moreover, the release characteristics are not always satisfactory.

It is therefore an object of the invention to provide a process for preparing microparticles laden with at least one active, in particular with at least one volatile organic active. The microparticles are to have good release properties. In particular, they are to release the active only after a period of latency. More particularly, it is desirable to achieve controlled release of the active. For example, it may be desirable for the release rates to be very substantially constant over a prolonged period. The laden microparticles are to be producible in a simple process and with high yield but without significant agglomeration or even destruction of the laden microparticles.

It has been found that, surprisingly, these and further objects are achieved by the process described hereinafter and the active-filled microparticles that are obtainable thereby.

The present invention therefore relates to a process for producing microparticles laden with at least one active, wherein the microparticles are formed from a thermoplastic organic, polymeric material and in the unladen state, in their interior, have at least one cavity connected via pores to the surface of the microparticles, wherein
(a) a composition of unladen microparticles are impregnated with a liquid comprising the active, whereby laden microparticles are obtained, which contain in the interior cavity the liquid, and
(b) subjecting the laden microparticles to thermal treatment by passing a stream of free flowing laden microparticles in a carrier gas through a heated zone at a temperature of at least 20 K, in particular at least 40 K, e.g. 20 to 300 K or 20 to 250 K, in particular 30 to 250 K or 30 to 200 K, especially 40 to 220 K or 40 to 200 K above the softening temperature of the thermoplastic organic polymeric material, where the average residence time of the laden microparticles in the heated zone is not more than 60 s, in particular not more than 30 s, especially not more than 20 s or not more than 10 s, e.g. in the range of 0.1 to 60 s, in particular in the range of 0.1 to 30 s, more particularly in the range of 0.5 to 20 s and especially in the range of 1 to 10 s.

The present invention further relates to compositions of microparticles filled with at least one active, in particular with at least volatile organic active, which is obtainable by a process of the invention, and to the use thereof, especially in a product selected from perfumes, washing and cleaning compositions, cosmetic compositions, personal care compositions, hygiene articles, foods, food supplements, fragrance dispensers and fragrances.

The present invention further relates to products comprising an inventive composition of microparticles filled with at least one active, in particular with at least one volatile organic active, and to the use thereof, especially for controlled release of actives of low molecular weight and specifically for controlled release of aroma chemicals.

The invention is associated with a number of advantages:
By the process of the invention the pores of the microparticles laden with the volatile organic are tightly sealed without significantly destroying or agglomerating the laden microparticles.
Despite the high temperatures the loss of the active during remains low during step b) due to the short residence time in the heated zone. This benefit allows in particular the inclusion of volatile organic actives into the microparticles.
As the pores of the laden microparticles are tightly sealed, they can be stored over a prolonged period without any significant loss of the active, which is in particular important in case of sensible or volatile organic actives.
By the process of the invention the laden microparticles are obtained as a free flowing powder which can be easily incorporated into.
The release of the active can be controlled in a simple manner, e.g. a spontaneous release can be achieved by applying mechanical forces to the laden microparticles.
The microparticles that are used as starting material are easily and inexpensively producible.
The process for loading is very versatile with regard to the feedstocks to be used and is especially suitable for a multitude of volatile organic actives.

The present invention relates more particularly to the following embodiments 1 to 30:
1. A process for producing microparticles laden with at least one volatile organic active, wherein the microparticles are formed from a thermoplastic organic, polymeric material and in the unladen state, in their interior, have at least one cavity connected via pores to the surface of the microparticles, wherein the above described steps (a) and (b) are taken.
2. The process according to embodiment 1, where the temperature in the heated zone is in the range from 130 to 350° C., in particular in the range from 140 to 300° C., especially in the range from 140 to 250° C.
3. The process according to any of the preceding embodiments, where the softening temperature of the thermoplastic organic polymeric material is in the range from 50 to 160° C., in particular in the range from 55 to 150° C.
4. The process according to any of the preceding embodiments, where the heating zone has a straight tubular geometry.
5. The process according to embodiment 4, where the heating zone is arranged in a downpipe.
6. The process according to any of the preceding embodiments, where the mass density of the stream of free flowing laden microparticles passed through the heated zone is in the range from 5 to 500 g/m³, in particular in the range from 10 to 200 g/m³, especially in the range from 20 to 200 g/m³.
7. The process according to any of the preceding embodiments, where the stream of free flowing laden microparticles exhibits an essentially laminar flow when passed through the heated zone.
8. The process according to any of the preceding embodiments, where the stream of free flowing laden microparticles is quenched immediately after leaving the heated zone.
9. The process according to any of the preceding embodiments, wherein the composition of unladen microparticles, prior to the impregnation, have an average particle diameter D[4,3] of 5 to 600 μm, as determined by light scattering.
10. The process according to embodiment 9, wherein at least 80% of those microparticles that have a particle diameter that differs from the average particle diameter of the microparticles in the composition by not more than 20% each have an average of at least 10 pores having a diameter at least 20 nm and at most 1/5 of the average particle diameter.
11. The process according to any of the preceding embodiments, wherein the thermoplastic organic, polymeric material comprises at least one polymer having a glass transition temperature or melting point in the range from 45 to 140° C.
12. The process according to any of the preceding embodiments, wherein the thermoplastic organic, polymeric material has a solubility in dichloromethane of at least 50 g/L at 25° C.
13. The process according to any of the preceding embodiments, wherein the thermoplastic organic, polymeric material comprises at least one aliphatic-aromatic polyester.
14. The process according to embodiment 13, wherein the aliphatic-aromatic polyester is an ester of an aliphatic dihydroxy compound esterified with a composition of aromatic dicarboxylic acid and aliphatic dicarboxylic acid.
15. The process according to embodiment 14, wherein the aliphatic-aromatic polyester is selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST).
16. The process according to any one of embodiments 13 to 15, wherein the thermoplastic organic, polymeric material, besides the aliphatic-aromatic polyester, additionally comprises at least one further polymer which is different from aliphatic-aromatic polyesters and which is in particular selected from the group consisting of aliphatic polyesters, polyanhydrides, polyesteramides, modified polysaccharides and proteins and mixtures thereof.
17. The process according to embodiment 16, wherein the further polymer is selected from the group consisting of polymerized hydroxycarboxylic acids, aliphatic-aliphatic copolyesters, polylactones, poly(p-dioxanones), polyanhydrides and aliphatic polyesteramides.
18. The process according to any one of embodiments 16 or 17, wherein the further polymer is selected from polylactic acid, aliphatic-aliphatic copolyesters, aliphatic poly-C₅-C₁₂-lactones and polyhydroxy fatty acids.
19. The process according to any one of embodiments 16 or 17, wherein the mass ratio of the aliphatic-aromatic polyester to the at least one further polymer which is different from aliphatic-aromatic polyesters is in the range from 30:70 to 99:1 or in the range from 30:70 to 80:20, in particular in the range from 35:65 to 75:25 and especially in the range form 40:60 to 70:30.
20. The process according to any of the preceding embodiments, wherein the active is liquid at 22° C. and 1013 mbar or has a melting point below 100° C.
21. The process according to any of the preceding embodiments, wherein the active is selected from aroma chemicals, organic active crop protection ingredients, organic active pharmaceutical ingredients, cosmetic actives, and actives for construction chemical applications.
22. The process according to embodiment 21, wherein the active is an aroma chemical which is liquid at 22° C. and 1013 mbar, or a mixture of aroma chemicals which is liquid at 22° C. and 1013 mbar.
23. The process according to embodiment 22, wherein the aroma chemical comprises at least one volatile fragrance.
24. A composition of microparticles filled with at least one active, obtainable by a process according to any of the preceding embodiments.
25. The composition according to embodiment 24, comprising the active in a total amount of 5% to 75% by weight, based on the total weight of the laden microparticles.
   26. The composition according to any one of embodiments 24 or 25 in the form of a powder.
27. A product comprising a composition according to any of embodiments 24 to 26 in a proportion by weight of 0.01% to 80% by weight based on the total weight of the product.
28. The product according to embodiment 27, wherein the product is selected from perfumes, washing products, cleaning products, cosmetic products, personal care products, hygiene articles, foods, food supplements, fragrance dispensers and fragrances.
29. The use of the composition according to any of embodiments 24 to 26 in a product selected from perfumes, washing and cleaning products, cosmetic products, personal care products, hygiene articles, foods, food supplements, fragrance dispensers and fragrances.
30. The use of the composition according to any of embodiments 24 to 26 for controlled release of actives.

Here and throughout this application the term “softening temperature” relates to the socalled Vicat softening temperature (VST), determined in accordance with DIN EN ISO 306:2014-03 at a heating rage of 50° C./h by applying a point load of 10 N to a specimen, so called VST/A50 temperature, if not stated otherwise.

Here and throughout this application the term “thermoplastic organic, polymeric material” relates to a material based on organic polymers, which is thermoplastic. A thermoplastic, or thermosoftening material is a polymer material that softens and thus becomes pliable or moldable upon heating to a certain elevated temperature and solidifies upon cooling below said certain temperature. In particular, the thermoplastic polymeric material is a material which does not undergo a chemical reaction upon heating to the temperature, where it softens and becomes pliable or moldable.

Here and throughout this application the term “active” is understood by the person skilled in the art to mean a chemical compound that triggers a physiological effect in living beings and plants, and substances that cause a chemical effect or catalyze a chemical reaction in inanimate nature. Examples of actives are aroma chemicals, organic crop protecting agents, organic pharmaceutical agents, cosmetic actives and actives for uses in the construction sector, called construction chemicals, especially catalysts for products in the construction sector, e.g. crosslinking or polymerization catalysts.

The term “volatile organic active” refers to an organic or organometallic chemical compound having a boiling temperature or evaporation temperature of at most 250° C. at 101.3 kPa and/or a vapour pressure at 20° C. of at least 50 Pa.

The term “organic active of low molecular weight” refers to an organic or organometallic chemical compound having a defined molecular weight Mn which is generally below 1000 daltons and typically in the range from 80 to <1000 daltons and especially in the range from 100 to 500 daltons.

The “molecular weight Mn” or the “molar mass Mn” is the number-average molecular weight or molar mass. The “molecular weight Mw” or the “molar mass Mw” is the massaverage molecular weight or molar mass. “Polydispersity” is as the ratio of weightaverage to number-average, i.e. the quotient Mw/Mn.

Unless stated otherwise, the term “room temperature” indicates a temperature of 22° C.

The term “biodegradable” is understood to mean that the substance in question, the unfilled microparticles here, in the test of OECD Guideline 301B from 1992 (measurement of evolution of CO₂ on composting in a mineral slurry and comparison with the theoretical maximum possible evolution of CO₂) after 28 days and 25° C. undergoes biodegradation of at least 5%, particularly at least 10% and especially at least 20%.

According to the invention, the unladen microparticles intended for impregnation are formed from a thermoplastic organic polymer material and have openings, called pores, on the particle surface. These pores are connected to one or more cavities in the interior of the microparticles, such that the respective liquid containing the active can penetrate into the cavity through the pores on impregnation of the microparticles. In this way, the microparticles are laden with the active present in the liquid. In other words, in the impregnation, the microparticles are treated with the respective liquid in such a way that the cavity present in the unfilled microparticles is largely or completely filled with the respective liquid and consequently laden with the active.

The walls of these cavities are formed by the thermoplastic organic polymer material. In other words, the organic polymer material surrounds the cavities that are present in the microparticles and are connected by the pores, and is therefore also referred to as polymeric wall material or wall material. In the unfilled state, these cavities comprise a gas or gas mixture, typically air, CO₂ or an inert gas such as nitrogen or argon, which is largely or completely displaced on impregnation of the unladen microparticles with the liquid that contains the active. The microparticles have one or more cavities in their interior. In the case of multiple cavities, the cavities may be separated from one another by the polymeric wall material or connected to one another. More particularly, the microparticles intended for loading have, in their interior, a multitude of mutually connected cavities, i.e. a network of cavities, connected by the pores in the surface of the microparticles.

The term “microparticles” means that the particles have dimensions in the micrometer range, i.e. below 1000 μm, particularly below 800 μm and especially below 600 μm. The value reported here is that value that exceeds 90% by volume of the particles present in a sample, which is also referred to as the D[v, 0.9] value. Typically, at least 90% by volume of the microparticles intended for loading have dimensions of at least 1 μm, particularly at least 2 μm and especially at least 5 μm (called the D[v, 0.1] value).

The microparticles intended for impregnation or loading preferably have an average particle diameter, i.e. a D[4,3] value, in the range of 5 to 600 μm, particularly in the range of 7 to 500 μm and especially in the range of 10 to 400 μm. In a first preferred embodiment, the average particle diameter D[4,3] is in the range of 5 to <100 μm, particularly in the range of 5 to 50 μm, especially in the range of 5 to 30 μm. In a second preferred embodiment, the average particle diameter D[4,3] is in the range of 30 to 600 μm, particularly in the range of 50 to 500 μm and especially in the range of 100 to 400 μm.

The microparticles intended for impregnation with the active preferably have a Sauter diameter, i.e. a D[3,2] value, in the range of 2.5 to 400 μm, particularly in the range of 3.5 to 250 μm and especially in the range of 5 to 200 μm.

The microparticles intended for impregnating preferably have a D[v, 0.5] value in the range of 3 to 500 μm, particularly in the range of 4 to 300 μm and especially in the range of 8 to 300 μm

Here and hereinafter, all figures for particle sizes, particle diameters and particle size distributions, including the D[v, 0.1], D[v, 0.5], D[v, 0.9], D[4,3] and D[3,2] values, are based on the particle size distributions ascertained by static laser light scattering to ISO 13320:2009 on samples of the microparticles. The abbreviation SLS is also used hereinafter for the expression “static laser light scattering to ISO 13320:2009”. In this connection, the D[v, 0.1] value means that 10% by volume of the particles of the measured sample have a particle diameter below the value reported as D[v, 01]. Accordingly, the D[v, 0.5] value means that 50% by volume of the particles of the measured sample have a particle diameter below the value reported as D[v, 0.5], and the D[v, 0.9] value means that 90% by volume of the particles of the measured sample have a particle diameter below the value reported as D[v, 0.9]. The D[4,3] value is the volume-weighted average determined by means of SLS, which is also referred to as the De Brouckere mean and corresponds to the mass average for the particles of the invention. The D[3, 2] value is the surface-weighted average determined by means of SLS, which is also referred to as the Sauter diameter.

The microparticles intended for impregnation are preferably regular-shaped particles, especially sphere-shaped particles. The term “regular-shaped” means that the surface of the particles, apart from the pores, does not have any great depressions in the wall material or elevations of wall material. The term “spherical” means that the particles have approximately the shape of a rotational ellipsoid and especially a spherical shape, where, in a particle in particular, the ratio of the longest axis through the center of the particle to the shortest axis through the center of the particle does not exceed a value of 2 and is especially in the range from 1:1 to 1.5:1.

The microparticles intended for impregnation are especially spherical microparticles that preferably have an average particle diameter D[4,3] of 5 to 600 μm, particularly of 7 to 500 μm and especially in the range of 10 to 400 μm. In a first preferred embodiment, the average particle diameter D[4,3] of the spherical microparticles is in the range of 5 to <100 μm, particularly in the range of 5 to 50 μm, especially in the range of 5 to 30 μm. In a second preferred embodiment, the average particle diameter D[4,3] of the spherical microparticles is in the range of 30 to 600 μm, particularly in the range of 50 to 500 μm and especially in the range of 100 to 400 μm.

Preferably, the microparticles intended for impregnation have at least 10, preferably at least 20, pores on their surface. Preferably, the diameter of the pores is in the range from 20 nm to 1/5 of the average particle diameter. The diameter of these pores is preferably at least 50 nm, more preferably at least 100 nm and especially at least 200 nm. The diameter of these pores will generally not exceed 20 μm, especially 10 μm, and is preferably in the range of 50 nm to 20 μm, in particular in the range of 100 nm to 20 μm and especially in the range of 200 nm to 10 μm, depending on the respective average particle diameter D[4,3].

Especially preferred are spherical microparticles intended for impregnation that have an average particle diameter D[4,3] in the range from 10 to 600 μm, particularly of 30 to 500 μm and especially of 50 to 400 μm, where at least 80% of those microparticles that have a particle diameter that differs from the average particle diameter of the microparticles in the composition by not more than 20% each have an average of at least 10 pores having a diameter in the range from 20 nm to 1/5 of the average particle diameter, and where the diameter of each of these pores is preferably at least 50 nm, more preferably at least 100 nm and especially at least 200 nm. The diameter of these pores will generally not exceed 20 μm, especially 10 μm, and is preferably in the range from 50 nm to 20 μm, in particular in the range from 100 nm to 20 μm and especially in the range from 200 nm to 10 μm.

The pore diameters given herein can be determined by means of scanning electron microscopy (Phenom Pro X). For this purpose, various close-up images are taken and these are retrospectively automatically measured using the ProSuite (FibreMetric) software from Phenom. The pores of a selected region of a particle are identified using the difference in contrast and the surfaces thereof are automatically measured. The diameter for each surface is calculated with the assumption that the surfaces are circular. (Sample size 100 pores). In the context of the evaluation, only those pores whose pore diameter is at least 20 nm are considered. Depending on the particle size, the images are recorded, for larger particles with 1600- to 2400-times magnification, and for smaller particles with up to 8000-times magnification. In order to determine the size of at least 10 pores, only those microparticles whose particle diameter does not deviate from the mean particle diameter of the composition of microparticles by more than 20% are taken into consideration.

The following assumptions are made for evaluation of the number of pores based on the total surface area of the microparticle: Since these are spherical particles, the image only shows half the surface of the particle. If the image of a microparticle shows at least 5 pores whose diameter is at least 20 nm and whose diameter is in the range from 1/5000 to 1/5 of the mean particle diameter, then the total surface comprises at least 10 pores.

The evaluation of the thus obtained data can be carried out according to the following procedure:
1. The mean particle diameter D[4,3] of the microparticles is already determined in the microparticle dispersion, using static light scattering. The upper and lower limits of the particle diameter of the microparticles which are taken into consideration for determining the pores (±20%) can be calculated from this.
2. The microparticle dispersion is dried.
3. From a sample, in each case 20 images showing multiple microparticles are taken by means of scanning electron microscopy.
4. 20 microparticles are selected, whose particle diameter is in the range ±20% of the mean particle diameter of the microparticles. The particle diameter thereof is thus measured with the ProSuite (FibreMetric) software from Phenom.
5. The pores of each of these 20 microparticles are measured. For this purpose, the surface areas of the visible pores are measured automatically and the diameter thereof is calculated.
6. The individual values of the pore diameters are checked as to whether their diameter met the condition of being in the range from 1/5000 to 1/5 of the mean particle diameter and being at least 20 nm.
7. The number of pores meeting this condition is determined and multiplied by two.
8. It is verified whether at least 16 microparticles have on average at least 10 pores.

According to the invention, the microparticles are formed from an organic polymeric material, which is thermoplastic. The organic polymeric material may in principle be any thermoplastic organic polymers as used in a known manner for production of porous, gas-filled microparticles or a mixture of thermoplastic organic polymers. The thermoplastic organic polymeric material may consist of one or more thermoplastic organic polymers but it may also contain small amounts of non-thermoplastic materials, e.g. duroplastic polymers, as long as these components do not impart the thermoplasticity of the organic polymeric material. In particular, the amount of non-thermoplastic material will not exceed 10% by weight, in particular 5% by weight of the total amount of thermoplastic organic polymeric material which forms the microparticles.

Examples of such polymeric wall materials are in particular condensation polymers such as polyesters, including aliphatic polyesters, semiaromatic polyesters and aromatic polyesters, and also polyamides, polyesteramides, polycarbonates, but also addition polymers, such as polystyrenes, polyacrylates, polyolefins, polyureas and polyurethanes, including polyesterurethanes and polyetherurethanes, and blends of the aforementioned polymers. Preferably, the wall material comprises at least one condensation polymer, especially at least one polyester.

According to the invention, the thermoplastic organic polymeric material, hereinafter also termed “wall material” softens at a certain elevated temperature, the so called softening temperature, precisely the Vicat softening temperature, also termed VST, especially the VST/A50. Preferably the softening temperature of the thermoplastic organic polymeric material is in the range from 50 to 160° C., in particular in the range from 55 to 150° C.

The softening temperatures of large number of thermoplastic organic polymers and polymer blends have been reported in the art. It can be easily determined in accordance with DIN EN ISO 306:2014-03 as described above. Apart from that it correlates with the glass transition temperature and/or with the melting temperature of a thermoplastic polymer. For blends it can be estimated by the so called Gordon-Taylor equation, the Couchman-Karasz equation, the Fox equation or the Kwei equation (see P. F. Fox, Advanced Dairy Chemistry Vol. 3, Springer Science and Business Media, 1992, p. 316; D. R. Heldman, Encyclopedia of Agricultural, Food, and Biological Engineering, CRC Press, 2003, p 760; T. G. Fox, Bull. Am. Phys. Soc. (1956), Vol. 1, p 123; M. F. Sonnenschein, Polyurethanes, John Wiley & Sons 2014, p 155 f. and the references cited therein).

Preferably, the wall material comprises at least one polymer having a glass transition temperature or melting temperature in the range from 45 to 160° C., in particular in the range from 50 to 150° C. If the polymer has a melting point, i.e. is semicrystalline or crystalline, it preferably has a melting or crystallization temperature in the range from 45 to 160° C., in particular in the range from 60 to 150° C. If the polymer is amorphous, it preferably has a glass transition temperature in the range from 45 to 160° C., in particular in the range from 50 to 150° C. The glass transition temperature here is typically determined by means of dynamic differential calorimetry (DSC) to DIN EN ISO 11357-1:2017-02. The melting temperature here is typically determined by means of dynamic differential calorimetry (DSC) to DIN EN ISO 11357-3:2018-07.

Preferably, the wall material has a solubility in dichloromethane of at least 50 g/L at 25° C.

Preferred wall materials comprise at least one semiaromatic polyester as main constituent. Semiaromatic polyesters are also referred to as aliphatic-aromatic polyesters, i.e. polyesters based on aromatic dicarboxylic acids and aliphatic dihydroxyl compounds, and polyesters based on mixtures of aromatic dicarboxylic acids with aliphatic dicarboxylic acids and aliphatic dihydroxyl compounds. Aliphatic-aromatic polyesters are preferably polyesters based on mixtures of aliphatic dicarboxylic acids with aromatic dicarboxylic acids and aliphatic dihydroxyl compound. These polymers may be present individually or in the mixtures thereof. Wall materials based on semiaromatic polyesters of this kind are typically biodegradable for the purposes of this invention, and hence so are the unfilled microparticles produced therefrom.

Preferably, “aliphatic-aromatic polyesters” shall also be understood to mean polyester derivatives such as polyetheresters, polyesteramides or polyetheresteramides and polyesterurethanes, as described, for example, in WO 2012/2013506. The suitable aliphatic-aromatic polyesters include linear, non-chain-extended polyesters, as described for example in WO 92/09654. Preference is given to chain-extended and/or branched aliphatic-aromatic polyesters. The latter are known from WO 96/15173, WO 96/15174, WO 96/15175, WO 96/15176, WO 96/21689, WO 96/21690, WO 96/21691, WO 96/21692, WO 96/25446, WO 96/25448 and WO 98/12242, to which explicit reference is made. Likewise considered are mixtures of different aliphatic-aromatic polyesters. Interesting recent developments are based on renewable raw materials and are described inter alia in WO 2006/097353, WO 2006/097354 and WO 2010/034710.

Particularly preferred aliphatic-aromatic polyesters include polyesters comprising as essential components:
A) an acid component formed from
   a1) 30 to 99 mol % of at least one aliphatic dicarboxylic acid or the ester-forming derivatives thereof or mixtures thereof,
   a2) 1 to 70 mol % of at least one aromatic dicarboxylic acid or the ester-forming derivative thereof or mixtures thereof and
B) at least one diol component selected from C₂- to C₁₂-alkanediols and
C) optionally a component selected from
   c1) a compound having at least three groups capable of ester formation,
   c2) a diisocyanate or polyisocyanate,
   c3) a diepoxide or polyepoxide.

Aliphatic dicarboxylic acids and the ester-forming derivatives thereof (a1) that are generally considered are those having 2 to 18 carbon atoms, preferably 4 to 10 carbon atoms. They may be either linear or branched. However, it is also possible in principle to employ dicarboxylic acids having a greater number of carbon atoms, for example having up to 30 carbon atoms.

Examples of aliphatic dicarboxylic acids and the ester-forming derivatives include: oxalic acid, malonic acid, succinic acid, 2-methylsuccinic acid, glutaric acid, 2-methylglutaric acid, 3-methylglutaric acid, α-ketoglutaric acid, adipic acid, pimelic acid, azelaic acid, sebacic acid, brassylic acid, fumaric acid, 2,2-dimethylglutaric acid, suberic acid, diglycolic acid, oxaloacetic acid, glutamic acid, aspartic acid, itaconic acid and maleic acid, their anhydrides and their C₁-C₄-alkyl esters. These dicarboxylic acids or the ester-forming derivatives thereof may be used individually or as a mixture of two or more thereof.

It is preferable to employ succinic acid, adipic acid, azelaic acid, sebacic acid, brassylic acid or their respective ester-forming derivatives or mixtures thereof. It is particularly preferable to employ succinic acid, adipic acid or sebacic acid or the respective ester-forming derivatives thereof or mixtures thereof. Succinic acid, azelaic acid, sebacic acid and brassylic acid additionally have the advantage that they are obtainable from renewable raw materials.

The aromatic dicarboxylic acids or the ester-forming derivatives thereof (a2) may be used individually or as a mixture of two or more thereof. Particular preference is given to using terephthalic acid or the ester-forming derivatives thereof such as dimethyl terephthalate.

Generally, the diols (B) are selected from branched or linear alkanediols having 2 to 12 carbon atoms, preferably 4 to 6 carbon atoms, or cycloalkanediols having 5 to 10 carbon atoms. Examples of suitable alkanediols are ethylene glycol, propane-1,2-diol, propane-1,3-diol, butane-1,2-diol, butane-1,4-diol, pentane-1,5-diol, 2,4-dimethyl-2-ethylhexane-1,3-diol, 2,2-dimethylpropane-1,3-diol, 2-ethyl-2-butylpropane-1,3-diol, 2-ethyl-2-isobutylpropane-1,3-diol, 2,2,4-trimethylhexane-1,6-diol, especially ethylene glycol, propane-1,3-diol, butane-1,4-diol and 2,2-dimethylpropane-1,3-diol (neopentyl glycol). Examples of suitable cylcoalkanediols are cyclopentanediol, cyclohexane-1,4-diol, cyclohexane-1,2-dimethanol, cyclohexane-1,3-dimethanol, cyclohexane-1,4-dimethanol and 2,2,4,4-tetramethylcyclobutane-1,3-diol. The aliphatic-aromatic polyesters may also include combinations of different alkanediols or cycloalkanediols. Particular preference is given to butane-1,4-diol, especially in combination with adipic acid as component a1), and butane-1,4-diol or propane-1,3-diol, especially in combination with sebacic acid as component a1). Propane-1,3-diol also has the advantage that it is obtainable as a renewable raw material.

More preferably, the aliphatic-aromatic polyester is selected from polybutylene azelateco-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST). The aforementioned aliphatic-aromatic polyesters have VST/A50 values in the range of 50 to 160° C., in particular in the range of 55 to 150° C.

The preferred aliphatic-aromatic polyesters are characterized by a molecular weight Mn in the range from 1000 to 100 000 g/mol, especially in the range from 9000 to 75 000 g/mol, preferably in the range from 10 000 to 50 000 g/mol.

Preferably, at least one of the polymers forming the wall material has a glass transition temperature or a melting point in the range from 45 to 160° C., in particular in the range from 50 to 150° C.

In a preferred group of embodiments, the wall material of the microparticles consists essentially of an aliphatic-aromatic polyester. In this group of embodiments the wall material of the microparticles consists in particular to an extent of at least 95% by weight, especially to an extent of at least 99% by weight, based on the wall material of an aliphatic-aromatic polyester.

Preferably, the aliphatic-aromatic polyester has a glass transition temperature or a melting point in the range from 45 to 160° C., in particular in the range from 50 to 150° C.

In a more preferred group of embodiments, the wall material of the microparticles comprises a combination of an aliphatic-aromatic polyester. In this group of embodiments the wall material of the microparticles consists in particular to an extent of at least 95% by weight, especially to an extent of at least 99% by weight, based on the wall material, of the combination of at least one aliphatic-aromatic polyester and at least one further polymer.

In particular, the wall material comprises a combination of at least one aliphatic-aromatic polyester and also at least one further thermoplastic polymer selected from polymers that are not aliphatic-aromatic polyesters. Preferably, the amount of aliphatic-aromatic polyester in the mixture amounts to at least 30% of the combination, e.g. from 50 to 99% of the combination. In particular, the mass ratio of the aliphatic-aromatic polyester to the at least one further polymer which is different from aliphatic-aromatic polyesters is in the range from 30:70 to 99:1 or in the range from 30:70 to 80:20, in particular in the range from 35:65 to 75:25 and especially in the range form 40:60 to 70:30.

The further polymer is usually a thermoplastic polymer. Preferably, the further polymer, which is not an aliphatic-aromatic polyester, has a glass transition temperature or a melting point in the range from 45 to 160° C., in particular in the range from 50 to 150° C.

Examples of said further polymer that is not an aliphatic-aromatic polyester and which is thermoplastic include but are not limited to: polyacrylates, polyamides, polycarbonates, polystyrenes, aliphatic polyesters, in particular aliphatic-aliphatic copolyesters, polyetheresters, polyanhydrides, polyesteramides, polylactones, aromatic/aromatic polyesters, polyolefines, polyureas, polyurethanes, modified polysaccharides and proteins.

The further polymer is preferably selected from the group consisting of aliphatic polyesters, aliphatic polyanhydrides, aliphatic polyetheresters, aliphatic polyesteramides, modified polysaccharides and proteins and mixtures thereof. In particular the further polymer is selected from the group consisting of polyesters of hydroxycarboxylic acids, aliphatic-aliphatic polyesters, polylactones, poly(p-dioxanones), polyanhydriden and aliphatic polyesteramides. The at least one further polymer is especially selected from the group consisting of aliphatic polyesters, especially polylactic acid, polyhydroxy fatty acids, aliphatic-aliphatic polyesters, polyhydroxy acetic acids and poly-C₆-C₁₂-lactones and mixtures thereof.

In a particular group of embodiments the further polymer comprises at least one aliphatic polyester, which is selected from the group consisting of polyhydroxy acetic acids and polylactic acid and copolymers thereof. Amongst these, polylactic acids, also termed polylactides (also termed PLA) and copolymers of lactic acid (PLA copolymers), in particular copolymers of lactic acid and glycolic acid, i.e. polylactid-co-glycolid (also termed PLGA). Amongst PLA and PLA-copolymers PLA is preferred. Polylactic acid having a molecular weight of 30 000 to 120 000 daltons and a glass transition temperature (Tg) in the range from 50 to 65° C. is particularly suitable. Most preferably, amorphous polylactic acid having a proportion of D-lactic acid greater than 9% is used.

Preferred components in the mixtures with the at least one aliphatic-aromatic polyester are polyhydroxyacetic acid, PLA copolymers (polylactide and polylactic acid copolymers) and PLGA copolymers, and here especially polylactide copolymers. Polylactic acid having a molecular weight of 30 000 to 120 000 daltons and a glass transition temperature (Tg) in the range from 50 to 65° C. is particularly suitable. Most preferably, amorphous polylactic acid having a proportion of D-lactic acid greater than 9% is used. Polyhydroxyalkanoates are understood to mean primarily poly-4-hydroxybutyrates and poly-3-hydroxybutyrates, and also include copolyesters of the aforementioned hydroxybutyrates with 3-hydroxyvalerates (P(3HB)-co-P(3HV)) or 3-hydroxyhexanoate.

According to a further preferred group of embodiments the further polymer comprises an aliphatic polyester of the group of polyhydroxy fatty acids. Polyhydroxy fatty acids are polyesters based on hydroxy fatty acids, which have from 1 to 18, in particular from 1 to 6 carbon atoms between the carbon atom bearing the OH group and the carbon atom of the carboxyl group. Polyhydroxy fatty acids also include polyesters of 2-hydroxybutyric acid, in particular their homopolymers. Accordingly, polylactic acid and polyhydroxyacetic acid are not polyhydroxy fatty acids. Typically, polyhydroxy fatty acid comprise repeating monomer units of the formula (1b)

[—O—CHR—(CH₂)ₘ—CO—] (1a)

[—O—CHR′—CO—] (1b)

where R is hydrogen or a linear or branched alkyl group having 1 to 20, preferably 1 to 16 carbon atoms, especially 1 to 6 carbon atoms, R′ is a linear or branched alkyl group having 2 to 20, preferably 2 to 16 carbon atoms, preferably 2 to 6 carbon atoms and m=numbers from 1 to 18, preferably 1, 2, 3, 4, 5 and 6.

Polyhydroxy fatty acid include homopolymers (also termed homopolyesters), i.e. polyhydroxy fatty acids of identical types of hydroxy fatty acid monomers and copolyesters, i.e. polyhydroxy fatty acids of different types of hydroxy fatty acid monomers. Polyhydroxy fatty acids can be used in arbitrary mixtures of polyhydroxy fatty acids.

Polyhydroxy fatty acid usually have a weight average molecular weight M_{w} in the range of 5000 to 1 000 000, in particular in the range of 30 000 to 1 000 000, or in the range of 70 000 to 1 000 000, preferably in the range of 100 000 to 1000 000 or in the range of 300 000 to 600 000 and/or having melting temperatures in the range of 100 to 180° C.

In one embodiment of the invention, the at least one polyhydroxy fatty acid is selected from the group consisting of
poly-3-hydroxypropionates (P3H P);
polyhydroxybutyrates (PHB);
polyhydroxyvalerates (PHV);
polyhydroxyhexanoates (PHHx);
polyhydroxyoctanoates (PHO);
polyhydroxyoctadecanoates (PHOD);
copolyesters of hydroxybutyric acid and at least one monomer selected from the group consisting of 3-hydroxypropionic acid, hydroxyvaleric acid, hydroxyhexanoic acid, hydroxyoctanoic acid and hydroxyoctadecaniuc acid;
copolyesters of hydroxyvaleric acid with at least one monomer selected from the group consisting of 3-hydroxypropionic acid, hydroxyhexanoic acids, hydroxyoctanoic acids and hydroxyoctadecanoic acids;
copolyesters of hydroxyhexanoic acid with at least one monomer selected from the group consisting of 3-hydroxypropionic acid, hydroxyoctanoic acid and hydroxyoctadecanoic acid;
Poly-C₆-C₁₂-lactones, especially polycaprolactone.

Suitable polyhydroxybutyrates (PHB) may be selected from the group consisting of poly(2-hydroxybutyrates) (P2HB), poly(3-hydroxybutyrates) (P3HB), poly(4-hydroxybutyrates) (P4HB) and copolymers of at least 2 hydroxybutyric acids selected from the group consisting of 2-hydroxybutyric acid, 3-hydroxybutyric acid and 4-hydroxybutyric acid. Further suitable are copolymers of 3-hydroxybutyric acid and 4-hydroxybutyric acid. These copolymers are characterized by the following abbreviations: [P(3HB-co-4HB)], where 3HB is 3-hydroxybutyrate and 4HB is 4-hydroxybutyrates.

Poly(3-hydroxybutyrates) are marketed for example by PHB Industrial under the brand Biocycle® and by Tianan under the name Enmat®. Poly-3-hydroxybutyrate-co-4-hydroxybutyrates are known from Metabolix in particular. They are sold under the trade name Mirel®.

Suitable polyhydroxyvalerates (PHV) may be selected from the group consisting of homopolymers of 3-hydroxyvaleric acid [=poly(3-hydroxyvalerates) (P3HV)], homopolymers of 4-hydroxyvaleric acid [=poly(4-hydroxyvalerates) (P4HV)]; homopolymers of 5-hydroxyvaleric acid [=poly(5-hydroxyvalerates) (P5HV)]; homopolymers of 3-hydroxymethylvaleric acid [=poly(3-hydroxymethylvalerates) (P3MHV)]; copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid.

Suitable polyhydroxyhexanoates (PHHx) may be selected from the group consisting of poly(3-hydroxyhexanoates) (P3HHx), poly(4-hydroxyhexanoates) (P4HHx), poly(6-hydroxyhexanoates) (P6HHx) and copolymers of at least 2 hydroxyhexanoic acids selected from the group consisting of 3-hydroxyhexanoic acid, 4-hydroxyhexanoic acid and 6-hydroxyhexanoic acid.

Suitable polyhydroxyoctanoates (PHO) may be selected from the group consisting of poly(3-hydroxyoctanoates) (P3HO), poly(4-hydroxyoctanoates) (P4HO), poly(6-hydroxyoctanoates) (P6HO) and copolymers of at least 2 hydroxyoctanoic acids selected from the group consisting of 3-hydroxyoctanoic acid, 4-hydroxyoctanoic acid and 6-hydroxyoctanoic acid.

Suitable copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids may be selected from the group consisting of
copolyesters of 4-hydroxybutyric acid with 3-hydroxyvaleric acid [P(4HB-co-3HV)]
copolyesters of 3-hydroxybutyric acid with 3-hydroxyvaleric acid [P(3HB-co-3HV)]
copolyesters of 4-hydroxybutyric acid with 3-hydroxyhexanoic acid [P(4HB-co-3HHx)]
copolyesters of 3-hydroxybutyric acid with 3-hydroxyhexanoic acid [P(3HB-co-3HHx)]
copolyesters of 4-hydroxybutyric acid with 3-hydroxyoctanoic acid [P(4HB-co-3HO)] and
copolyesters of 3-hydroxybutyric acid with 3-hydroxyoctanoic acid [P(3HB-co-3HO)]
copolyesters of 4-hydroxybutyric acid with 3-hydroxyoctadecanoic acid [P(4HBco-3HOD)] and
copolyesters of 3-hydroxybutyric acid with 3-hydroxyoctadecanoic acid [P(3HBco-3HOD)]

Preference is given to using poly-3-hydroxybutyrate-co-3-hydroxyhexanoate having a 3-hydroxyhexanoate proportion of 1 to 20 and preferably of 3 to 15 mol % based on the total amount of polyhydroxy fatty acid. Such poly-3-hydroxybutyrate-co-3-hydroxyhexanoates [P(3HB-co-3HHx] are known from Kaneka and are commercially available under the trade names Aonilex™ X131A and Aonilex™ X151A.

Suitable copolyesters of hydroxyvaleric acid are preferably copolyesters of 4-hydroxyvaleric acid and/or 3-hydroxyvaleric acid with at least one monomer selected from the group consisting of 3-hydroxypropionic acid, hydroxyhexanoic acids, hydroxyoctanoic acids, especially 3-hydroxyoctanoic acid and hydroxyoctadecanoic acids.

Suitable copolyesters of hydroxyhexanoic acid are preferably copolyesters of 3-hydroxyhexanoic acid with at least one monomer selected from the group consisting of 3-hydroxypropionic acid and hydroxyoctanoic acid, preferably 3-hydroxyoctanoic acid and hydroxyoctadecanoic acids.

According to a particular preferred group of embodiments the further polymer comprises an aliphatic polyester of the group of polylactones, in particular from the group of poly-C₆-C₁₂-lactones, esepcially polycaprolactones (PCL). Polylactones refer to polyesters obtainable by ring-opening polymerization of lactones, in particular C₆-C₁₂-lactones, especially epsilon-caprolactone (ε-caprolactone). Polycaprolactones are therefore polyhydroxy fatty acids with repeating monomer units of the general formula (1) [—O—CHR—(CH₂)ₘ—CO—], in which m is 4 to 10, in case of caprolactone m=4, and R is hydrogen. In the context of the invention, the term polycaprolactone is understood to mean both homopolymers of epsilon-caprolactone and copolymers of epsilon-caprolactone. Suitable copolymers are, for example, copolymers of epsilon-caprolactone with monomers selected from the group consisting of lactic acid, lactide, hydroxyacetic acid and glycolide. Polycaprolactones are marketed, for example, by Perstorp under the brand name Capa™ or by Daicel under the brand name Celgreen™.

In a preferred embodiment, the at least one polyhydroxy fatty acid is a polycaprolactone.

In one group of embodiments of the invention, the at least one polyhydroxy fatty acid is selected from the group consisting of
poly(3-hydroxypropionates) (P3HP);
polyhydroxybutyrates (PHB);
polyhydroxyvalerates (PHV);
polyhydroxyhexanoates (PHHx);
polyhydroxyoctanoates (PHO);
polyhydroxyoctadecanoates (PHOD);
copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids;
copolyesters of hydroxyvaleric acid with at least one monomer selected from the group consisting of 3-hydroxypropionic acid, hydroxyhexanoic acids, hydroxyoctanoic acids and hydroxyoctadecanoic acids;
copolyesters of hydroxyhexanoic acid with at least one monomer selected from the group consisting of 3-hydroxypropionic acid, hydroxyoctanoic acid and hydroxyoctadecanoic acid;
polycaprolactones;

excluding poly(4-hydroxybutyrates) and poly(3-hydroxybutyrates), furthermore copolyesters of the aforementioned hydroxybutyrates with 3-hydroxyvalerates (P(3HB)-coP(3HV)) or 3-hydroxyhexanoate.

In one embodiment of the invention, the at least one polyhydroxy fatty acid is selected from the group consisting of poly(3-hydroxypropionates) (P3H P); poly(2-hydroxybutyrates) (P2HB); copolymers of at least 2 hydroxybutyric acids selected from the group consisting of 2-hydroxybutyric acid, 3-hydroxybutyric acid and 4-hydroxybutyric acid; copolymers of 3-hydroxybutyric acid and 4-hydroxybutyric acid; poly(3-hydroxyvalerates) (P3HV); poly(4-hydroxyvalerates) (P4HV); poly(5-hydroxyvalerates) (PSHV); poly(3-hydroxymethylvalerates) (P3MHV); copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid; poly(3-hydroxyhexanoates) (P3HHx); poly(4-hydroxyhexanoates) (P4HHx); poly(6-hydroxyhexanoates) (P6HHx); copolymers of at least 2 hydroxyhexanoic acids selected from the group consisting of 3-hydroxyhexanoic acid, 4-hydroxyhexanoic acid and 6-hydroxyhexanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; copolyesters of 2-hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids; copolyesters of 4-hydroxybutyric acid with 3-hydroxyoctanoic acid [P(4HB-co-3HO)], copolyesters of 3-hydroxybutyric acid with 3-hydroxyoctanoic acid [P(3HB-co-3HO)], copolyesters of 4-hydroxybutyric acid with 3-hydroxyoctadecanoic acid [P(4HB-co-3HOD)], copolyesters of 3-hydroxybutyric acid with 3-hydroxyoctadecanoic acid [P(3HB-co-3HOD)]; copolyesters of hydroxyvaleric acid, especially of 3-hydroxyvaleric acid or 4-hydroxyvaleric acid with at least one monomer selected from the group consisting of 3-hydroxypropionic acid, hydroxyhexanoic acids, hydroxyoctanoic acids and hydroxyoctadecanoic acids; copolyesters of 3-hydroxyhexanoic acid with at least one monomer selected from the group consisting of 3-hydroxypropionic acid, hydroxyoctanoic acid, preferably 3-hydroxyoctanoic acid and hydroxyoctadecanoic acids; and polycaprolactones.

According to a further group of embodiments, the further polymer of the wall material comprises at least one polyhydroxy fatty acid, which is selected from the group consisting of polyhydroxy alkanoates. Polyhydroxy alkanoates mainly refer to poly-4-hydroxybutyrates and poly-3-hydroxybutyrates and copolyesters of the aforementioned hydroxybutyrates with 3-hydroxyvalerates (P(3HB)-co-P(3HV)) or 3-hydroxyhexanoates. Usually, polyhydroxy alkanoates have a weight average molecular weight Mw in the range of 30 000 to 1 000 000 g/mol and in particular in the range of 100 000 to 600 000 g/mol.

According to a further group of embodiments, the further polymer of the wall material comprises at least one aliphatic-aliphatic polyester. Aliphatic-aliphatic polyesters are understood to mean polyesters based on aliphatic dicarboxylic acids and aliphatic dihydroxyl compounds, and polyesters based on mixtures of aliphatic dicarboxylic acids with aliphatic dicarboxylic acids and aliphatic dihydroxyl compounds.

Examples of aliphatic carboxylic acids which are suitable for the preparation of aliphatic-aliphatic polyesters are the aliphatic dicarboxylic acids mentioned under (a1), especially those having 2 to 18 carbon atoms, preferably 4 to 10 carbon atoms. Preference is given to aliphatic-aliphatic polyesters in which the aliphatic dicarboxylic acid is selected from succinic acid, adipic acid, azelaic acid, sebacic acid, brassylic acid and mixtures thereof. Particular preference is given to succinic acid, adipic acid and sebacic acid and mixtures thereof. To prepare the aliphatic-aliphatic polyesters, instead of the dicarboxylic acids, their respective ester-forming derivatives or mixtures thereof with the dicarboxylic acids may also be used.

Examples of aliphatic diols which are suitable for the preparation of the aliphatic-aliphatic polyesters are the diols mentioned as component (B), for example branched or linear alkanediols having 2 to 12 carbon atoms, preferably 4 to 6 carbon atoms, or cycloalkanediols having 5 to 10 carbon atoms. Examples of suitable alkanediols are ethylene glycol, 1,2-propanediol, 1,3-propanediol, 1,2-butanediol, 1,4-butanediol, 1,5-pentanediol, 2,4-dimethyl-2-ethylhexane-1,3-diol, 2,2-dimethyl-1,3-propanediol, 2-ethyl-2-butyl-1,3-propanediol, 2-ethyl-2-isobutyl-1,3-propanediol, 2,2,4-trimethyl-1,6-hexanediol, especially ethylene glycol, 1,3-propanediol, 1,4-butanediol and 2,2-dimethyl-1,3-propanediol (neopentyl glycol). Examples of cycloalkanediols are cyclopentanediol, 1,4-cyclohexanediol, 1,2-cyclohexanedimethanol, 1,3-cyclohexanedimethanol, 1,4-cyclohexanedimethanol and 2,2,4,4-tetramethyl-1,3-cyclobutanediol. The aliphatic-aliphatic polyesters may also comprise mixtures of different alkanediols condensed in. Particular preference is given to 1,4-butanediol, especially in combination with one or two aliphatic dicarboxylic acids selected from succinic acid, adipic acid and sebacic acid, as component a1).

Examples of particularly preferred aliphatic-aliphatic polyesters are polybutylene succinate adipate, polybutylene succinate, polybutylene sebacate and polybutylene succinate sebacate.

The preferred aliphatic-aliphatic polyesters often have a molecular weight Mn in the range from 1000 to 100 000 g/mol, particularly in the range from 2000 to 75 000 g/mol, especially in the range from 5000 to 50 000 g/mol.

According to a further group of embodiments the further polymer of the wall material comprises at least one poly-p-dioxanone (poly-1,4-dioxan-2-one). Poly-p-dioxanones (poly-1,4-dioxan-2-one) refer to poly(ether-esters) obtainable by ring-opening polymerization of 1,4-dioxan-2-one. In the context of the present invention, the term poly(p-dioxanones) are understood to mean homopolymers of 1,4-dioxan-2-one, which have the general structural unit [—O—CH₂—CH₂—O—CH₂—CO—]ₙ. In the context of the present invention, the term poly(p-dioxanones) is also understood to mean copolymers of 1,4-dioxan-2-one with lactone monomers. Particularly suitable are copolymers of 1,4-dioxan-2-one with at least one further monomer selected from the group consisting of glycolide, lactide and epsilon-caprolactone.

According to a further group of embodiments the further polymer of the wall material comprises at least one polyanhydride. Polyanhydrides refer to polymers having the general structural unit

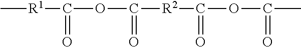

as characteristic base units of the main chain. R¹ and R² can be the same or different aliphatic or aromatic radicals. Suitable polyanhydrides are described in Kumar et al, Adv. Drug Delivery Reviews 54 (2002), pp. 889-910. Particularly suitable are the polyanhydrides described in Kumar et al. Adv. Drug Delivery Reviews 54 (2002), on p. 897, which is fully incorporated here by way of reference. In one embodiment of the invention, the polyanhydride is selected from the group of aliphatic polyanhydrides, especially from the group consisting of polysebacic acid and polyadipic acid.

A further group of polymers, which can be used in combination with the aliphatic-aromatic polyester as wall material, are polyester amides, in particular aliphatic polyester amides. Aliphatic polyesteramides are copolymers bearing both amide and ester functions. Suitable aliphatic polyesteramides are particularly polyesteramides obtained by polycondensation of ε-caprolactam, adipic acid, 1,4-butanediol and hexamethylene diamine, and polyesteramides obtained by polycondensation of adipic acid, 1,4-butanediol, diethylene glycol and hexamethylene diamines. Polyesteramides are marketed, for example, under the trade name BAK™ from Bayer, such as BAK™1095 or BAK™ 2195 for example.

A further group of polymers, which can be used in combination with the aliphatic-aromatic polyester as wall material, are polysaccharides. Polysaccharides are macromolecules in which a relatively large number of sugar residues are glycosidically linked to one another. Suitable polysaccharides in accordance with the invention are polysaccharides having a solubility in dichloromethane at 25° C. of at least 50 g/L. In the context of the invention, polysaccharides also include derivatives thereof if they have a solubility in dichloromethane at 25° C. of at least 50 g/L.

Suitable polysaccharides in accordance with the invention are preferably selected from the group consisting of modified starches such as, in particular, starch ethers and esters, cellulose derivatives such as, in particular, cellulose esters and cellulose ethers, chitin derivatives and chitosan derivatives.

Cellulose derivatives generally refer to celluloses chemically modified by polymeranalogous reactions. They comprise both products in which exclusively the hydroxyl hydrogen atoms of the glucose units of the cellulose have been substituted by organic or inorganic groups and those in which formally an exchange of the entire hydroxyl group has been effected (e.g. desoxycelluloses). Also products which are obtained from intramolecular elimination of water (anhydrocelluloses), oxidation reactions (aldehyde-, keto- and carboxycelluloses) or cleavage of the C₂,C₃-carbon bond of the glucose units (dialdehyde- and dicarboxycelluloses) are counted as cellulose derivatives. Finally, cellulose derivatives are also accessible by reactions such as crosslinking or graft copolymerization reactions. Since for all these reactions to some extent a multiplicity of reagents can be used and, in addition, the degree of substitution and polymerization of the cellulose derivatives obtained can be varied, an extensive palette of soluble and insoluble cellulose derivatives having markedly differing properties is known.

Suitable cellulose ethers are, for example, methyl cellulose, ethyl cellulose, hydroxyethyl cellulose, hydroxypropyl cellulose and hydroxypropylmethyl cellulose.

Suitable cellulose ethers are methylhydroxy-(C₁-C₄)-alkylcelluloses. Methylhydroxy(C₁-C₄)alkyl celluloses are understood to mean methyl hydroxy(C₁-C₄)alkyl celluloses of a wide variety of degrees of methylation and also degrees of alkoxylation.

The preferred methyl hydroxy(C₁-C₄)alkyl celluloses have an average degree of substitution DS of 1.1 to 2.5 and a molar degree of substitution MS of 0.03 to 0.9.

Suitable methyl hydroxy(C₁-C₄)alkyl celluloses are for example methyl hydroxyethyl cellulose or methyl hydroxypropyl cellulose.

Suitable cellulose esters are, for example, the esters of cellulose with C₂-C₄ monocarboxylic acids, such as cellulose acetate (commercially available from Eastmann CA-398-3), cellulose butyrate, cellulose acetobutyrate, cellulose propionate and cellulose acetopropionate. Cellulose esters are obtainable in a wide variety of degrees of polymerization and substitution.

A further group of polymers, which can be used in combination with the aliphatic-aromatic polyester as wall material, are proteins. Proteins to be used in accordance with the invention comprise polypeptides (acid amide-like condensation products of amino acids linked by peptide bonds) and derivatives thereof having a solubility in dichloromethane at 25° C. of at least 50 g/l. They polypeptides may be of natural or synthetic origin.

According to particularly preferred groups of embodiments, the wall material comprises a combination comprising or consisting of
i) at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) at least one aliphatic polyester, selected from the group consisting of polyhydroxy fatty acids, including in particular poly-C₆-C₁₂-lactones, polyhydroxy acetic acid, polylactic acid, and aliphatic-aliphatic polyesters, and mixtures thereof, especially from the group consisting of polylactides, aliphatic-aliphatic polyesters, poly-C₆-C₁₂-lactones and mixtures thereof.

According to especially preferred groups of embodiments, the wall material comprises a combination comprising or consisting of
i) at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) at least one aliphatic polyester, selected from the group consisting of polycaprolactones, of polylactic acid (PLA), polylactid glycolid, polybutylene succinate adipate, polybutylene succinate, polybutylene sebacate and polybutylene succinate sebacate.

Preference is given in accordance with the invention to mixtures of at least one aliphatic-aromatic polyester with one or more polymers that are not aliphatic-aromatic polyesters, with a proportion by weight of the aromatic-aliphatic polyester of 30% to 99% by weight, based on the total weight of aliphatic-aromatic polyester and the polymer that is not an aliphatic-aromatic polyester. Preferably, the proportion of the aliphatic-aromatic polyester is 30% to 80% by weight, preferably 35% to 75% by weight, further preferably 40% to 70% by weight, based on the total weight of the aliphatic-aromatic polyester and the further polymer that is not aliphatic-aromatic polyester.

According to particularly preferred groups of embodiments the wall material comprises a combination comprising or consisting of
i) 30 to 80% by weight, preferably 35 to 75% by weight, more preferably 40 to 70% by weight and especially 45 to 70% by weight, in each case based on the total mass of the wall material, of at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) 20 to 70% by weight, preferably 25 to 65% by weight, more preferably 30 to 60% by weight and especially 30 to 55% by weight, in each case based on the total mass of the wall material, of at least one aliphatic polyester, selected from the group consisting of polyhydroxy fatty acids, including in particular poly-C₆-C₁₂-lactones, polyhydroxy acetic acid, polylactic acid, and aliphatic-aliphatic polyesters, and mixtures thereof, especially from the group consisting of polylactides, aliphatic-aliphatic polyesters, poly-C₆-C₁₂-lactones and mixtures thereof.

According to especially preferred groups of embodiments the wall material comprises a combination comprising or consisting of
i) 30 to 80% by weight, preferably 35 to 75% by weight, more preferably 40 to 70% by weight and especially 45 to 70% by weight, in each case based on the total mass of the wall material, of at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) 20 to 70% by weight, preferably 25 to 65% by weight, more preferably 30 to 60% by weight and especially 30 to 55% by weight, in each case based on the total mass of the wall material, of at least one aliphatic polyester, selected from the group consisting of polycaprolactones, of polylactic acid (PLA), polylactid glycolid, polybutylene succinate adipate, polybutylene succinate, polybutylene sebacate and polybutylene succinate sebacate.

Preference is given to mixtures of an aliphatic-aromatic polyester with a further polymer which is not an aliphatic-aromatic polyester, especially the mixtures with aliphatic-aliphatic polyesters, in which the melting point of the aliphatic-aromatic polyester is at least 10° C., preferably at least 20° C., above the melting point of the further polymer, or the glass transition temperature of the aliphatic-aromatic polyester is at least 10° C., preferably at least 20° C., above the glass transition temperature of the further polymer. If the further polymer is an amorphous compound, the melting point of the aliphatic-aromatic polyester is at least 10° C., preferably at least 20° C., above the glass transition temperature of the further polymer.

In a preferred embodiment, the preferred aliphatic-aromatic polyesters are characterized by a molecular weight Mn in the range from 1000 to 100 000 g/mol, further preferably in the range from 9000 to 75 000 g/mol, further preferably in the range from 10 000 to 50 000 g/mol.

Especially preferred microparticles for loading are the particles described in WO 2018/065481 and the prior European patent application 18166159.6, especially those described in the examples therein.

The person skilled in the art is able to produce the microparticles described in a manner known per se, for example by the procedure described in WO 2011/088229 or WO 2015/070172 and especially in WO 2018/065481 or in prior European patent application 18166159.6.

The microparticles intended for loading are typically produced by a process in which
a) a water-in-oil emulsion (w/o emulsion) is prepared from water or an aqueous solution of a pore former as discontinuous phase and a continuous phase comprising a solution of at least one polymer or polymer mixture suitable as a wall material, particularly comprising at least one polyester, especially at least one aliphatic-aromatic polyester, in a water-immiscible solvent,
b) the w/o emulsion obtained in a) is emulsified in water in the presence of a dispersant to give a water-in-oil-in-water emulsion (w/o/w emulsion) with droplets of average size 10-600 μm, and the water-immiscible solvent is removed at a temperature in the range from 20 to 80° C., preferably from 20 to 45° C.,
c) the microparticles formed in process step b) are separated off and optionally dried.

The microparticles are thus typically produced by removing the solvent in a w/o/w emulsion. In the first step, an emulsion of water droplets or droplets of the aqueous pore former solution is formed in the polyester solution. This w/o emulsion is in turn emulsified in water to obtain the w/o/w emulsion and the water-immiscible solvent is removed therefrom. Removal of the solvent makes the polymer or polymer mixture insoluble and it separates out at the surface of the water droplets or the aqueous pore former droplets. During this wall forming process, the pores are simultaneously formed, advantageously brought about by the pore former. Pore formers are, for example, compounds that release gas under the process conditions of step b).

Pore formers are typically agents that release a gas, e.g. CO₂, and are preferably selected from ammonium carbonate, sodium carbonate, ammonium hydrogencarbonate, ammonium sulfate, ammonium oxalate, sodium hydrogencarbonate, ammonium carbamate and sodium carbamate.

Further suitable pore formers are water-soluble low molecular weight compounds that create an osmotic pressure. Removal of the water-insoluble solvent, on account of the concentration gradient that exists between the inner aqueous droplets with pore former and the outer aqueous disperse phase, builds up a concentration gradient which leads to migration of the water in the direction of the inner droplets and hence to the formation of pores. Such pore formers are preferably selected from sugars such as monosaccharides, disaccharides, oligosaccharides and polysaccharides, urea, inorganic alkali metal salts such as sodium chloride and inorganic alkaline earth metal salts such as magnesium sulfate and calcium chloride. Particular preference is given to glucose and sucrose and urea.

Further suitable pore formers are polymers that are soluble in both phases, for example polyethylene glycol (PEG) and polyvinylpyrrolidone (PVP). Since these polymers are soluble in both phases, they migrate from the aqueous phase into the oil phase owing to diffusion.

The processes for producing the spherical microparticles always lead to a population of microparticles, and therefore the term “composition of spherical microparticles” is also used.

In a preferred embodiment, the composition of the microparticles intended for loading is produced by the double emulsion method. This more preferably comprises the process steps specified herein, a), b) and c). In this way, spherical microparticles having the abovementioned particle sizes and pore contents are obtained.

Process Step a)

For this purpose, the polymer or polymer mixture suitable as wall material is dissolved in a water-immiscible solvent.

In relation to process step a), “water-immiscible” means that the solvent has a solubility in water, at a temperature of 20° C. and a pressure of 1 bar, of ≤90 g/L. In addition, the water-immiscible solvent preferably has a boiling point of at least 30° C. According to the general knowledge of those skilled in the art, solvents are chemically inert to the substances to be dissolved therein; that is to say, they merely serve for dilution or dissolution. Free radically-polymerizable monomers are not solvents in the context of the invention.

Preference is given to aprotic non-polar and aprotic polar solvents or solvent mixtures, which have a water solubility of <90 g/L (at 20° C.). Preferred solvents are, for example, dichloromethane, chloroform, ethyl acetate, n-hexane, cyclohexane, methyl tert-butyl ether, pentane, diisopropyl ether and benzene, or mixtures of two or more of these solvents with one another. Dichloromethane is particularly preferred. Further suitable solvent mixtures are those that form an azeotrope having a boiling point within the range from 20 to 80° C. One example is the azeotrope of hexane and methyl ethyl ketone (MEK) in the weight ratio of 72:28.

In general, the polymer or polymer mixture suitable as wall material is used as a 1% to 50% by weight solution in the water-immiscible solvent. Preferably, the polymer solution thus prepared is a 2% to 30% by weight, especially 5% to 20% by weight, solution in the water-immiscible solvent.

According to the invention, an emulsion of a solution of at least one polymer suitable as wall material or of a polymer mixture is chosen. Preference is given to an emulsion of a solution of the polymer or polymer mixture suitable as wall material which is at least one polyester, particularly at least one aliphatic-aromatic polyester and especially a mixture of at least one aliphatic-aromatic polyester with a further polymer which is not an aliphatic-aromatic polyester and is especially an aliphatic-aliphatic polyester such as polylactic acid. If the wall material used is a mixture of polymers, the solution used to prepare the microparticles can be obtained by mixing the individual polymer solutions or be prepared by co-dissolving a mixture of polymers. The polymer or polymer mixture suitable as wall material is the wall material of the later microparticle. The wall material of the microparticles preferably has a solubility at 25° C. and 1 bar of at least 50 g/L in dichloromethane.

According to a preferred group of embodiments the continuous phase prepared in step a) consists essentially of the solution of the aliphatic-aromatic polyester in a in the water-immiscible solvent. In particular, the continuous phase consists to at least 95% by weight, in particular to at least 99% by weight, based on the weight of the continuous phase, of the solution of the aliphatic-aromatic polyester in a in the water-immiscible solvent.

According to a further preferred group of embodiments the continuous phase prepared in step a) comprises a combination of at least one aliphatic-aromatic polyester and at least one further polymer, which is not an aliphatic-aromatic polyester, and which is preferably selected from the group consisting of the aforementioned further polymers, which are mentioned as preferred or especially preferred and mixtures thereof, where the combination of these polymers is dissolved in the the water-immiscible solvent. In this solution, the mass ratio of the aliphatic-aromatic polyester and the at least one further polymer, which is not an aliphatic-aromatic polyester, is typically in the range of 30:70 to 99:1 or in the range of 30:70 to 80:20, in particular in the range of 35:65 to 75:25 and especially in the range of 40:60 to 70:30 or in the range of 30:70 to 70:30 or in the range of 45:55 to 70:30.

According to particularly preferred groups of embodiments the polymers used for preparing the continuous phase comprise a combination comprising or consisting of
iii) at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
iv) at least one aliphatic polyester, selected from the group consisting of polyhydroxy fatty acids, including in particular poly-C₆-C₁₂-lactones, polyhydroxy acetic acid, polylactic acid, and aliphatic-aliphatic polyesters, and mixtures thereof, especially from the group consisting of polylactides, aliphatic-aliphatic polyesters, poly-C₆-C₁₂-lactones and mixtures thereof.

According to especially preferred groups of embodiments the polymers used for preparing the continuous phase comprise a combination comprising or consisting of
iii) at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
iv) at least one aliphatic polyester, selected from the group consisting of polycaprolactones, of polylactic acid (PLA), polylactid glycolid, polybutylene succinate adipate, polybutylene succinate, polybutylene sebacate and polybutylene succinate sebacate.

According to particularly preferred groups of embodiments the polymers used for preparing the continuous phase comprise a combination comprising or consisting of
iii) 30 to 80% by weight, preferably 35 to 75% by weight, more preferably 40 to 70% by weight and especially 45 to 70% by weight, in each case based on the total mass of the wall material, of at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
iv) 20 to 70% by weight, preferably 25 to 65% by weight, more preferably 30 to 60% by weight and especially 30 to 55% by weight, in each case based on the total mass of the wall material, of at least one aliphatic polyester, selected from the group consisting of polyhydroxy fatty acids, including in particular poly-C₆-C₁₂-lactones, polyhydroxy acetic acid, polylactic acid, and aliphatic-aliphatic polyesters, and mixtures thereof, especially from the group consisting of polylactides, aliphatic-aliphatic polyesters, poly-C₆-C₁₂-lactones and mixtures thereof.

According to especially preferred groups of embodiments the polymers used for preparing the continuous phase comprise a combination comprising or consisting of
iii) 30 to 80% by weight, preferably 35 to 75% by weight, more preferably 40 to 70% by weight and especially 45 to 70% by weight, in each case based on the total mass of the wall material, of at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
iv) 20 to 70% by weight, preferably 25 to 65% by weight, more preferably 30 to 60% by weight and especially 30 to 55% by weight, in each case based on the total mass of the wall material, of at least one aliphatic polyester, selected from the group consisting of polycaprolactones, of polylactic acid (PLA), polylactid glycolid, polybutylene succinate adipate, polybutylene succinate, polybutylene sebacate and polybutylene succinate sebacate.

Water or an aqueous solution of the pore former is emulsified in this polymer solution in process step a).

The aqueous solution of the pore former is preferably a 0.1% to 10% by weight aqueous solution of the pore former, especially of a pore former selected from ammonium hydrogencarbonate and ammonium carbonate. Particular preference is given to using ammonium carbonate, especially a 0.1% to 1% by weight solution of ammonium carbonate in water, as pore former solution.

0.1 to 10 parts by weight of the pore former, based on the 100 parts by weight of the sum total of the polymers that form the wall material, are used. The polymers that form the wall material consist of preferably at least one polyester or a mixture of two or more polyesters, or at least one polyester and at least one non-polyester polymer, especially of at least one aliphatic-aromatic polyester, at least one additional polymer other than an aliphatic-aromatic polyester, e.g. an aliphatic polyester, and optionally at least one further polymer. Preference is given to using 1 to 5 parts by weight, especially 1.3 to 3 parts by weight, of the pore former based on 100 parts by weight of the sum total of the polymers that form the wall material.

The emulsifying in process step a) is usually effected with the aid of a disperser, for example a rotor-stator or rotor-rotor disperser, or with the aid of a high-pressure disperser or high-pressure homogenizer or of an ultrasound homogenizer or of a gear dispersing machine. More particularly, the aforementioned homogenizing or dispersing machines are suitable for production of the w/o emulsion, since these can introduce high shear energy into the system and hence small droplet sizes are obtained. The average droplet size, i.e. the D[4,3] value, of the emulsion droplets is generally 0.2 to 50 μm.

The w/o emulsion produced in process step a) can optionally be stabilized with one or more dispersants. Dispersants suitable for w/o emulsions are common knowledge and are mentioned for example in EP 2794085 and EP 3007815, the teaching of which is expressly incorporated by reference.

To prepare the w/o emulsion in step a) and for stabilization thereof, instead of or together with the aforementioned dispersants, one or more emulsifiers can be used preferably having an HLB value according to Griffin in the range of 2 to 10, especially in the range of 3 to 8. The HLB value (HLB=hydrophilic lipophilic balance) according to Griffin (W. C. Griffin: Classification of surface-active agents by HLB. In: J. Soc. Cosmet. Chem. 1, 1949, pp. 311-326) is a dimensionless number between 0 and 20 which provides information on the water and oil solubility of a compound. Preferably, these are non-ionic emulsifiers having an HLB value according to Griffin in the range of 2 to 10, particularly in the range of 3 to 8. However, also suitable are anionic and zwitterionic emulsifiers having an HLB value according to Griffin in the range of 2 to 10, particularly in the range of 3 to 8.

Such emulsifiers are generally used in an amount from 0.1 to 10% by weight, especially 0.5 to 5% by weight, based on the total weight of the emulsion prepared in step a). In general, the emulsifier or emulsifiers are added to the solution of the polymer or polymers in the water-immiscible solvent before emulsifying water or the aqueous solution of the pore former into this solution.

Examples of suitable emulsifiers having an HLB value according to Griffin in the range of 2 to 10 are:
sorbitan fatty acid esters, particularly sorbitan mono-, di- and trifatty acid esters and mixtures thereof, such as sorbitan monostearate, sorbitan monooleate, sorbitan monolaurate, sorbitant tristearate, sorbitan sesquioleate, sorbitan dioleate, sorbitan trioleate;
fatty acid esters of glycerol or of polyglycerol such as glycerol monostearate, glycerol distearate, glycerol monooleate, glycerol dioleate, glycerol monostearate monoacetate, glycerol monoacetate monooleate, polyglycerol polyrinoleate (E476), e.g. the commercially available emulsfier PGPR 90;
lactyl esters of fatty acid monoesters of glycerol;
lecithins;
ethoxylated castor oils, ethoxylated hydrogenated castor oils with degrees of ethoxylation in the range of 2 to 20;
ethoxylated and/or propoxylated C₁₂-C₂₂-alkanols having degrees of alkoxylation in the range of 2 to 10, e.g. stearyl alcohol ethoxylate having a degree of ethoxylation in the range of 2 to 5, stearyl alcohol ethoxylate-co-propoxylate having degrees of alkoxylation in the range of 2 to 8, isotridecyl ethoxylates having degrees of ethoxylation in the range of 2 to 3 and isotridecyl ethoxylateco-propoxylates with degrees of alkoxylation in the range of 2 to 5;
ethoxylated and/or propoxylated C₄-C₁₆-alkylphenols having degrees of alkoxylation in the range of 2 to 10, e.g. nonylphenol ethoxylate having degrees of ethoxylation in the range of 2 to 5 and octylphenol ethoxylate having degrees of ethoxylation in the range of 2 to 5.

Process Step b)

The emulsification of the w/o-emulsion in water to give the w/o/w emulsion in process step b) is effected by stirring or shearing in the presence of a dispersant. It is possible here to meter an aqueous solution of the dispersant into the w/o emulsion. The dispersant is preferably initially charged in the form of an aqueous solution and the w/o emulsion is metered in. Depending on the energy input, it is possible to control the droplet size. Furthermore, the dispersant described below influences the size of the emulsion droplets in equilibrium.

The concentration of the dispersant in the aqueous solution is typically in the range from 0.1 to 8.0% by weight, in particular in the range from 0.3 to 5.0% by weight and especially in the range from 0.5 to 4.0% by weight, based on the total weight of the dispersant solution.

The weight ratio of the w/o emulsion prepared in step a) to water, preferably in the form of the aqueous solution of the dispersant, is typically in the range from 15:85 to 55:45, in particular in the range from 25:75 to 50:50, and especially in the range from 30:70 to 45:55.

The amount of dispersant applied in step b) is typically at least 0.1% by weight, in particular at least 0.2% by weight, based on the total weight of the w/o/w-emulsion, and is in particular in the range from 0.1 to 2% by weight, and especially in the range from 0.2 to 1% by weight, based on the total weight of the w/o/w-emulsion.

Larger droplets having an average droplet size D[4,3] in the range from 100 to 600 μm are obtained by means of customary stirrers, whereas average droplet sizes D[4,3] of below 100 μm are achieved by means of apparatuses for generating a high shear field. It is also possible to introduce sufficient shear energy by vigorous stirring that average droplet sizes with D[4,3] values in the range from 1 to <100 μm, preferably of 5 to 50 μm, are achieved. Should even higher shear energy input be intended, it may be advantageous to use apparatuses for generating a high shear field.

Suitable stirrer types include propeller stirrers, impeller stirrers, disk stirrers, paddle stirrers, anchor stirrers, pitched-blade stirrers, cross-beam stirrers, helical stirrers, screw stirrers and others.

Suitable apparatuses for generating a high shear field are comminutors operating by the rotor-stator principle, such as toothed ring dispersing machines, and also colloid and corundum disk mills and high-pressure and ultrasound homogenizers. Preference is given to the use of the toothed ring dispersing machines operating by the rotor-stator principle for generating the shear field. The diameter of the rotors and stators is typically in the range between 2 and 40 cm, depending on machine size and dispersing performance. The speed of rotation of such dispersing machines is generally in the range from 500 to 20 000 rpm (revolutions per minute), depending on the construction type. Of course, machines with large rotor diameters rotate at the lower end of the rotation speed range, while machines with small rotor diameters are usually operated at the upper end of the rotation speed range. The distance of the rotating parts from the stationary parts of the dispersing tool is generally 0.1 to 3 mm.

In a preferred embodiment, the final size of the emulsion droplets of the w/o/w emulsion should be an average diameter D[4,3] (determined by means of light scattering) of 100 to 600 μm. This final size is generally achieved just by stirring.

In a likewise preferred embodiment, the final size of the emulsion droplets of the w/o/w emulsion should have an average diameter of 10 to 100 μm, preferably 10 to 30 μm. This final size is typically achieved by means of shearing.

The shear energy introduced can be directly derived from the power consumption of the apparatus for generating a shear field, taking account of the heat loss. Thus, the shear energy input into the w/o/w emulsion is preferably 250 to 25 000 watts h/m³ batch size. Particular preference is given to an energy input of 500 to 15 000, especially 800 to 10 000, watts h/m³ batch size, calculated based on the motor current.

The w/o/w emulsion is produced in the presence of at least one dispersant. It is possible to produce the w/o/w emulsion in a combination of different dispersants but also possible to use a single dispersant. Suitable dispersants are, for example, cellulose derivatives such as hydroxyethyl cellulose, methyl hydroxyethyl cellulose, methyl cellulose and carboxymethyl cellulose, polyvinylpyrrolidone, copolymers of vinylpyrrolidone, gelatin, gum arabic, xanthan, casein, polyethylene glycols, and partly or completely hydrolyzed polyvinyl acetates (polyvinyl alcohols), and also methyl hydroxypropyl cellulose, and also mixtures of the above. Preferred dispersnats are partly or completely hydrolyzed polyvinyl acetates (polyvinyl alcohols) and also methyl hydroxy(C₁-C₄)alkyl celluloses as well as mixtures thereof. Particular preference is given to partially hydrolysed polyvinyl acetates, also termed partially hydrolysed polyvinyl alcohols (PVAs) with particularly having a degree of hydrolysis of 79% to 99.9%. In addition, PVA copolymers, as described in WO 2015/165836, are also suitable.

Methyl hydroxy(C₁-C₄)alkyl celluloses are understood to mean methyl hydroxy(C₁-C₄)alkyl celluloses of a wide variety of degrees of methylation and also degrees of alkoxylation.

The preferred methyl hydroxy(C₁-C₄)alkyl celluloses have an average degree of substitution DS of 1.1 to 2.5 and a molar degree of substitution MS of 0.03 to 0.9.

Suitable methyl hydroxy(C₁-C₄)alkyl celluloses are for example methyl hydroxyethyl cellulose or methyl hydroxypropyl cellulose.

A particularly preferred dispersant is methyl hydroxypropyl cellulose.

A particularly preferred dispersant is selected from the group of partially hydrolysed polyvinyl alcohols with particular preference given to with particular preference given to those having a degree of hydrolysis of 79% to 99.9%. An especially preferred dispersant is a carboxy modified anionic PVA having 1 to 6 mol-% of carboxyl groups, based on the amount of repeating units and a degree of hydrolysis of 85% to 90%. Amongst these, those are preferred, whose 4% by weight aqueous solution has a viscosity of 20.0 to 30.0 mPa*s at 20° C.

In order to stabilize the w/o/w emulsion, the dispersant is added to the aqueous phase. The concentration of the dispersant in the aqueous phase is typically in the range from 0.1 to 8.0% by weight, in particular in the range from 0.3 to 5.0% by weight and especially in the range from 0.5 to 4.0% by weight, based on the total weight of the aqueous phase. The weight ratio of the w/o emulsion prepared in step a) to the aqueous phase containing the dispersant is typically in the range from 15:85 to 55:45, in particular in the range from 25:75 to 50:50, and especially in the range from 30:70 to 45:55.

In a preferred embodiment, carboxy-modified anionic PVA (having a degree of hydrolysis of 85 to 90 mol % and proportion of carboxyl groups of 1 to 6 mol % whose 4% by weight aqueous solution has a viscosity of 20.0 to 30.0 mPa*s at 20° C.) is used as 0.1% to 5.0% by weight aqueous solution. Particular preference is given to aqueous solutions having a PVA content of 0.3% to 2.5% by weight, especially solutions having a PVA content of 0.5% to 1.5% by weight.

In a preferred process variant, in process step b), the emulsification to give the w/o/w emulsion is effected with a stirrer at a stirring speed of 5000 to 15 000 rpm over a period of 1 to 30 minutes. The droplets produced thereby have a mean diameter of 0.2 to 30 μm.

In a further preferred process variant, the emulsion is prepared at a stirring speed of 100 to 1000 rpm over a period of 1 to 30 minutes. The average diameter of the droplets produced thereby is preferably 100 to 600 μm.

During the emulsification, and optionally thereafter, the mixture is kept at a temperature in the range from 10 to 80° C. The temperature of the mixture is preferably selected such that it is below the glass transition temperature of the lowest softening amorphous polymer or below the melting point of the lowest melting crystalline polymer of the composition that forms the wall material. Higher temperatures are possible, but they may lead to partial closure of the pores over too long a period. The mixture is preferably kept at a temperature in the range from 20 to 45° C., especially from 20 to <40° C. Optionally, a vacuum may additionally be applied. For example, a vacuum in the range of 600 to 800 mbar or below 200 mbar may be applied. The measures, i.e. the stirring/shearing, the temperature and the optionally applied vacuum result in the evaporation of the water-immiscible organic solvent and the microparticles being left behind in the form of an aqueous dispersion.

Provided that the solvent is one having a vapor pressure ≥450 hPa at 20° C., it is sufficient to stir the w/o/w emulsion obtained in b) at room temperature, 20° C. Depending on the amount of the solvent and the ambient temperature, such an operation lasts for a few hours. Depending on the solvent, it is possible to facilitate the removal of the solvent by raising the temperature to a temperature of up to 80° C. and/or by applying a vacuum.

In the course of the removal of the water-immiscible solvent, pore formation is observed in the walls of the microparticles.

The microparticles formed by removal of the water-immiscible solvent are removed in process step c) and preferably dried. “Dried” is understood to mean that the microparticles comprise a residual amount of water of ≤5% by weight, preferably ≤1% by weight, based on the microparticles. The drying may for example be carried out in a stream of air and/or by applying a vacuum, optionally in each case with heating. This is accomplished, depending on the size of the microparticles, by means of convective driers such as spray driers, fluidized bed and cyclone driers, contact driers such as pan driers, paddle driers, contact belt driers, vacuum drying cabinet or radiative driers such as infrared rotary tube driers and microwave mixing driers.

One feature of the spherical microparticles thus obtained is that they are easy to fill, for example by suspending them in a solution.

A specifically preferred process for producing the microparticles is described in WO 2018/065481.

In the process of the invention, the microparticles are laden with at least one organic active.

Preferably, the organic active of low molecular weight is liquid at 22° C. and 1013 mbar or has a melting point below 100° C. Preference is given particularly to actives that are liquid at 22° C. and 1013 mbar.

In the process of the invention, it is possible to use either one active or a mixture of actives. This may be a mixture of actives from one class or a mixture of actives from different classes.

In the process of the invention, the microparticles may also be laden with a mixture of different actives.

In a preferred group of embodiments, the organic active of low molecular weight is an aroma chemical, especially an aroma chemical which is liquid at 22° C. and 1013 mbar or a mixture of two or more aroma chemicals which is liquid at 22° C. and 1013 mbar. Preferred aroma chemicals are hydrophobic and, especially at 25° C., have a water solubility in deionized water of not more than 100 mg/L.

The term “aroma chemical” is understood by the person skilled in the art to mean organic compounds usable as “odorant” and/or as “flavoring”. In the context of the present invention, “odorant” is understood to mean natural or synthetic substances having intrinsic odor. In the context of the present invention, “flavoring” is understood to mean natural or synthetic substances having intrinsic flavor. In the context of the present invention, “odor” or “olfactory perception” is the interpretation of the sensory stimuli which are sent from the chemoreceptors in the nose or other olfactory organs to the brain of a living being. The odor can be a result of sensory perception of the nose of odorant, which occurs during inhalation. In this case, the air serves as odor carrier.

Preferred aroma chemicals for loading of the microparticles are selected, for example, from the following compounds:

alpha-hexylcinnamaldehyde, 2-phenoxyethyl isobutyrate (Phenirat¹), dihydromyrcenol (2,6-dimethyl-7-octen-2-ol), methyl dihydrojasmonate (preferably having a cis isomer content of more than 60% by weight) (Hedione⁹, Hedione HC⁹), 4,6,6,7,8,8-hexamethyl-1,3,4,6,7,8-hexahydrocyclopenta[g]benzopyran (Galaxolide³), tetrahydrolinalool (3,7-dimethyloctan-3-ol), ethyl linalool, benzyl salicylate, 2-methyl-3-(4-tert-butylphenyl)propanal (Lilial²), cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styrenyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super³), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarbox-aldehyde (Lyral³), alpha-amylcinnamaldehyde, ethylene brassylate, (E)- and/or (Z)-3-methylcyclopentadec-5-enone (Muscenone⁹), 15-pentadec-11-enolide and/or 15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹⁰), 2-isobutyl-4-methyltetrahydro-2H-pyran-4-ol (Florol⁹), 2-ethyl-4-(2,2,3-trimethyl-3-cyclopenten-1-yl)-2-buten-1-ol (Sandolene¹), cis-3-hexenyl acetate, trans-3-hexenyl acetate, trans-2-cis-6-nonadienol, 2,4-dimethyl-3-cyclohexenecarboxaldehyde (Vertocitral¹), 2,4,4,7-tetramethyloct-6-en-3-one (Claritone¹), 2,6-dimethyl-5-hepten-1-al (Melonal²), borneol, 3-(3-isopropylphenyl)butanal (Florhydral²), 2-methyl-3-(3,4-methylenedioxyphenyl)propanal (Helional³), 3-(4-ethylphenyl)-2,2-dimethylpropanal (Florazon¹), tetrahydro-2-isobutyl-4-methyl-2H-pyran (Di hydrorosenon⁴), 1,4-bis(ethoxymethyl)cyclohexane (Vertofruct⁴), L-isopulegol (1R,2S,5R)-2-isopropenyl-5-methylcyclohexanol, pyranyl acetate (2-isobutyl-4-methyltetrahydropyran-4-yl acetate), nerol ((Z)-2,6-dimethyl-2,6-octadien-8-ol), neryl acetate, 7-methyl-2H-1,5-benzodioxepin-3(4H)-one (Calone¹⁹⁵¹⁵), 3,3,5-trimethylcyclohexyl acetate (preferably with a content of cis isomers of 70% by weight) or more and 2,5,5-trimethyl-1,2,3,4,4a,5,6,7-octahydronaphthalen-2-ol (Ambrinol S¹), tetrahydro-4-methyl-2-(2-methylpropenyl)-2H-pyran (rose oxide), 4-methyl-2-(2-methylpropyl)oxane or 4-methyl-2-(2-methylpropyl)-2H-pyran (Dihydrorosan⁴), prenyl acetate (=3-methylbut-2-enyl acetate), isoamyl acetate, dihydromyrcenol (2,6-dimethyloct-7-en-2-ol) and methylheptenone (6-methylhept-5-en-2-one) and mixtures thereof, and also mixtures thereof with one or more other aromas.

In the context of the present invention, the aforementioned aromas or odorants are accordingly preferably combined with mixtures of the invention.

If trade names are specified above, these refer to the following sources:

¹ trade name of Symrise GmbH, Germany;

² trade name of Givaudan AG, Switzerland;

³ trade name of International Flavors & Fragrances Inc., USA;

⁴ trade name of BASF SE;

⁵ trade name of Danisco Seillans S. A., France;

⁹ trade name of Firmenich S. A., Switzerland;

¹⁰ trade name of PFW Aroma Chemicals B. V., the Netherlands.

More particularly, the advantages of the invention are manifested in the case of aroma chemicals that are selected from volatile fragrances and aroma mixtures comprising at least one volatile fragrance. Volatile fragrances are understood to mean fragrances having a high vapor pressure at room temperature. A fragrance is considered to be a volatile fragrance especially when it has the following property: If a droplet of the volatile fragrance is applied to a strip of paper and left to evaporate off under ambient conditions at room temperature (22° C.), its odor is no longer perceptible to an experienced perfumer no longer than 2 hours after application. The volatile fragrances especially include the following compounds: rose oxide (tetrahydro-4-methyl-2-(2-methylpropenyl)-2H-pyran), 4-methyl-2-(2-methylpropyl)oxane or 4-methyl-2-(2-methylpropyl)2H-pyran (Dihydrorosan®), prenyl acetate (=3-methylbut-2-enyl acetate), isoamyl acetate, dihydromyrcenol (2,6-dimethyloct-7-en-2-ol) and methylheptenone (6-methylhept-5-en-2-one). If an aroma mixture comprising at least one volatile fragrance is used for loading, the proportion of the volatile fragrance is generally at least 1% by weight, especially at least 5% by weight, for example 1% to 99% by weight, especially 5% to 95% by weight, based on the total weight of the aroma chemical mixture used for loading.

Further odorants or aroma chemicals with which the odorants mentioned can be combined to give an odorant composition can be found, for example, in S. Arctander, Perfume and Flavor Chemicals, Vol. I and II, Montclair, N. J., 1969, Author's edition or K. Bauer, D. Garbe and H. Surburg, Common Fragrance and Flavor Materials, 4th. Ed., Wiley-VCH, Weinheim 2001. Specifically, the following may be mentioned:

extracts from natural raw materials such as essential oils, concretes, absolutes, resins, resinoids, balsams, tinctures, for example

ambra tincture; amyris oil; angelica seed oil; angelica root oil; anise oil; valerian oil; basil oil; tree moss absolute; bay oil; mugwort oil; benzoin resin; bergamot oil; beeswax absolute; birch tar oil; bitter almond oil; savory oil; bucco leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camellia oil blue; camellia oil roman; carrot seed oil; cascarilla oil; pine needle oil; spearmint oil; cumin oil; labdanum oil; labdanum absolute; labdanum resin; lavandin absolute; lavandin oil; lavender absolute; lavender oil; lemon grass oil; lovage oil; lime oil distilled; lime oil pressed; linalool oil; litsea cubeba oil; laurel leaf oil; macis oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; oregano oil; palmarosa oil; patchouli oil; perilla oil; Peruvian balsam oil; parsley leaf oil; parsley seed oil; petitgrain oil; peppermint oil; pepper oil; allspice oil; pine oil; poley oil; rose absolute; rosewood oil; rose oil; rosemary oil; sage oil dalmatian; sage oil Spanish; sandalwood oil; celery seed oil; spike lavender oil; star anise oil; styrax oil; tagetes oil; fir needle oil; tea tree oil; turpentine oil; thyme oil; tolu balsam; tonka absolute; tuberose absolute; vanilla extract; violet leaf absolute; verbena oil; vetiver oil; juniper berry oil; wine yeast oil; vermouth oil; wintergreen oil; ylang oil; hyssop oil; civet absolute; cinnamon leaf oil; cinnamon bark oil; and fractions thereof or ingredients isolated therefrom.

Individual odorants are, for example, those from the group of
the hydrocarbons, for example 3-carene; alpha-pinene; beta-pinene; alphaterpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols, for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof, for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; (E/Z)-1-(1-methoxypropoxy)-3-hexene; the aliphatic ketones and oximes thereof, for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
the aliphatic sulfur-containing compounds, for example 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles, for example 2-nonenenitrile; 2-undecenenitrile; 2-tridecenenitrile; 3,12-tridecadienenitrile; 3,7-dimethyl-2,6-octadienenitrile; 3,7-dimethyl-6-octenenitrile;
the esters of aliphatic carboxylic acids, for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-ylacetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; (E/Z)-ethyl 2,4-decadienoate;
methyl 2-octynoate; methyl 2-nonynoate; allyl 2-isoamyloxyacetate; methyl 3,7-dimethyl-2,6-octadienoate; 4-methyl-2-pentyl crotonate;
the acyclic terpene alcohols, for example geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
the acyclic terpene aldehydes and ketones, for example geranial; neral; citronellal; 7-hydroxy-3,7-dimethyloctanal; 7-methoxy-3,7-dimethyloctanal; 2,6,10-trimethyl-9-undecenal; geranyl acetone; and also the dimethyl and diethyl acetals of geranial, neral, 7-hydroxy-3,7-dimethyloctanal; the cyclic terpene alcohols, for example menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones, for example menthone; isomenthone; 8-mercaptomenthan-3-one; carvone; camphor; fenchone; alpha-ionone; betaionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);
the cyclic alcohols, for example 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols, for example alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers, for example cineol; cedryl methyl ether; cyclododecyl methyl ether; 1,1-dimethoxycyclododecane; 1,4-bis(ethoxymethyl)cyclohexane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones, for example 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; cis-3-methylpent-2-en-1-ylcyclopent-2-en-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; cyclohexadec-5-en-1-one; 6,7-dihydro-1,1,2,3,3-pentamethyl-4(5H)-indanone; 8-cyclohexadecen-1-one; 7-cyclohexadecen-1-one; (7/8)-cyclohexadecen-1-one; 9-cycloheptadecen-1-one; cyclopentadecanone; cyclohexadecanone;
the cycloaliphatic aldehydes, for example 2,4-dimethyl-3-cyclohexenecarbaldehyde; 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
the cycloaliphatic ketones, for example 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols, for example 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or -6-indenyl acetate; 4,7-methano-3a,4,5,6,7,7a-hexahydro-5- or -6-indenyl propionate; 4,7-methano-3a,4,5,6,7,7a-hexahydro-5- or -6-indenyl isobutyrate; 4,7-methanooctahydro-5- or -6-indenyl acetate;
the esters of cycloaliphatic alcohols, for example 1-cyclohexylethyl crotonate; the esters of cycloaliphatic carboxylic acids, for example allyl 3-cyclohexylpropionate; allyl cyclohexyloxyacetate; cis- and trans-methyl dihydrojasmonate; cis- and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
the araliphatic alcohols, for example benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
the esters of araliphatic alcohols and aliphatic carboxylic acids, for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers, for example 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
the aromatic and araliphatic aldehydes, for example benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropyl phenyl)propanal; 2-methyl-3-(4-tert-butylphenyl) propanal; 2-methyl-3-(4-isobutylphenyl)propanal; 3-(4-tert-butylphenyl) propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxy-benzaldehyde; 4-hydroxy-3-ethoxybenzaldehyde; 3,4-methylenedioxybenzaldehyde; 3,4-di methoxybenzaldehyde; 2-methyl-3-(4-methoxyphenyl)propanal; 2-methyl-3-(4-methylenedioxyphenyl) propanal;
the aromatic and araliphatic ketones, for example acetophenone; 4-methylacetophenone; 4-methoxyacetophenone; 4-tert-butyl-2,6-di methylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl) ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;
the aromatic and araliphatic carboxylic acids and esters thereof, for example benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds, for example 2,4,6-trinitro-1,3-dimethyl-5-tert-butylbenzene; 3,5-dinitro-2,6-dimethyl-4-tert-butylacetophenone; cinnamonitrile; 3-methyl-5-phenyl-2-pentenonitrile; 3-methyl-5-phenylpentano-nitrile; methyl anthranilate; methyl N-methylanthranilate; Schiff's bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3-isopropylpyrazine; 2-isobutyl-3-methoxypyrazine;
the phenols, phenyl ethers and phenyl esters, for example estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;
the heterocyclic compounds, for example 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
the lactones, for example 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

In addition, suitable aroma chemicals are macrocyclic carbaldehyde compounds as described in WO 2016/050836.

Particular preference is given to mixtures of L-menthol and/or DL-menthol, L-menthone, L-menthyl acetate, or L-isopulegol, which are highly sought-after as analogs or substitutes for what are referred to as synthetic dementholized oils (DMOs). The mixtures of these minty compositions are preferably used in the ratio of L-menthol or DL-menthol 20-40% by weight, L-menthone 20-40% and L-menthyl acetate 0-20%, or in the ratio of 20-40% by weight, L-menthone 20-40% and L-isopulegol 0-20%.

The aforementioned aromas and aroma mixtures can be used as such or in a solvent which in itself is not an aroma. Typical solvents for aromas are especially those having a boiling point at standard pressure above 150° C. and which do not dissolve the wall material, e.g. diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-alkyl esters of aliphatic, aromatic or cycloaliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl 1,2-cyclohexanedicarboxylate, and dialkyl adipates, such as dibutyl adipate (e.g. Cetiol® B from BASF SE), C₈-C₂₂ fatty acid triglycerides, e.g. vegetable oils or cosmetic oils such as octanoyl/decanoyltriglyceride (e.g. the commercial product Myritol® 318 from BASF SE), dimethyl sulfoxide and white oils.

In a further group of embodiments, the organic active of low molecular weight is an active pharmaceutical ingredient, API for short. Active pharmaceutical ingredients are typically active therapeutic ingredients, active diagnostic ingredients and active prophylactic ingredients, and corresponding combinations of active ingredients. The active pharmaceutical ingredient(s) may be in an amorphous state, a crystalline state or a mixture thereof. The active pharmaceutical ingredient(s) may be labelled with a detectable label such as a fluorescent label, a radioactive label or an enzymatic or chromatographically detectable species, and be used as a mixture with this label for loading of the microparticles.

The API may have a high water solubility, for example a water solubility in deionized water of more than 10 mg/mL at 25° C. It is also possible to use active pharmaceutical ingredients having low water solubility as actives, for example those having a water solubility in deionized water of less than 10 mg/mL at 25° C.

Preferred active therapeutic, diagnostic and prophylactic ingredients are those APIs that are suitable for parenteral administration. Representative examples of suitable APIs are the following categories and examples of APIs and alternative forms of these APIs, such as alternative salt forms, free acid forms, free base forms and hydrates: analgesics/antipyretics; antiasthmatic drugs; antibiotics; antidepressants; antidiabetic drugs; antiphlogistics/inflammation inhibitors; antihypertensives; inflammation inhibitors; antineoplastics; antianxiety drugs; immunosuppressants; antimigraine drugs; tranquilizers/hypnotics; antitanginal drugs; antipsychotic drugs; antimanic drugs; antiarrhythmics; antiarthritic drugs; antigout drugs; anticoagulants; thrombolytic drugs; antifibrinolytic drugs; hemorheological drugs; antiplatelet drugs/thrombocyte aggregation inhibitors; anticonvulsives; anti-Parkinson's drugs; antihistamines/antipruritics; drugs for calcium regulation; antibacterial drugs; antiviral drugs; antimicrobial drugs; antiinfectives; bronchodilators; corticosteroids; steroidal compounds and hormones; hypoglycemic drugs; hypolipedemic drugs; proteins; nucleic acids; drugs useful for the stimulation of erythropoiesis; antiulcer drugs/antireflux drugs; antinausea drugs/antimosis drugs; oil-soluble vitamins and other medicaments.

Suitable active pharmaceutical ingredients are mentioned, for example, in WO 2007/070852, especially on pages 15 to 19. In addition, suitable active ingredients and drugs are listed in Martindale: The Extra Pharmacopoeia, 30th edition, The Pharmaceutical Press, London 1993.

In a further group of embodiments, the organic active of low molecular weight is an organic crop protecting agent. Organic crop protecting agents for loading of the microparticles are, for example, pesticides, especially selected from the group consisting of fungicides, insecticides, nematicides and herbicides, but also safeners, and growth regulators which for loading of the microparticles also as mixtures, for example as mixtures of two or more herbicides, mixtures of two or more fungicides, mixtures of two or more insecticides, mixtures of insecticides and fungicides, mixtures of one or more herbicides with a safener, and mixtures of one or more fungicides with a safener.

Typically, the pesticides are liquid or solid at 20° C. and 1013 mbar and are normally nonvolatile. The vapor pressure is typically below 0.1 mbar at 20° C., especially below 0.01 mbar. Crop protecting agents particularly suitable for loading are hydrophobic and, especially at 25° C., have a water solubility in deionized water of not more than 10 g/L and especially not more than 1 g/L.

Crop protecting agents are known to those skilled in the art, for example from The Pesticide Manual, 17th edition, The British Crop Protection Council, London, 2015. Suitable crop protecting agents are listed, especially, in WO 2018/019629 on pages 10 to 15.

Examples of suitable insecticides are compounds from the classes of the carbamates, organophosphates, organochlorine insecticides, phenylpyrazoles, pyrethroids, neonicotinoids, spinosins, avermectins, milbemycins, juvenile hormone analogs, alkyl halides, organotin compounds, nereistoxin analogs, benzoylureas, diacylhydrazines, METI acaricides, and unclassified insecticides such as chloropicrin, pymetrozine, flonicamid, clofentezin, hexythiazox, etoxazole, diafenthiuron, propargite, tetradifon, chlorofenapyr, DNOC, buprofezine, cyromazine, amitraz, hydramethylnon, acequinocyl, fluacrypyrim, rotenone, or the agriculturally acceptable salts and derivatives thereof.

Examples of suitable fungicides are compounds from the classes of the dinitroanilines, allylamines, anilinopyrimidines, antibiotic fungicides, aromatic hydrocarbons, benzenesulfonamides, benzimidazoles, benzisothiazoles, benzophenones, benzothiadiazoles, benzotriazines, benzyl carbamates, carbamates, carboxamides, carboxylic acid diamides, chloronitriles, cyanoacetamide oximes, cyanoimidazoles, cyclopropanecarboxamides, dicarboximides, dihydrodioxazines, dinitrophenyl crotonates, dithiocarbamates, dithiolanes, ethylphosphonates, ethylaminothiazolecarboxamides, guanidines, hydroxy(2-amino)pyrimidines, hydroxyanilides, imidazoles, imidazolinones, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothioates, phthalamic acids, phthalimides, piperazines, piperidines, propionamides, pyridazinones, pyridines, pyridinylmethylbenzamides, pyrimidineamines, pyrimidines, pyrimidinone hydrazones, pyrroloquinolinones, quinazolinones, quinolines, quinones, sulfamides, sulfamoyltriazoles, thiazolecarboxamides, thiocarbamates, thiophanates, thiophenecarboxamides, toluamides, triphenyltin compounds, triazines, triazoles and the agriculturally acceptable salts and derivatives thereof.

Examples of suitable fungicides are compounds from the classes of the acetamides, amides, aryloxyphenoxypropionates, benzamides, benzofurans, benzoic acids, benzothiadiazinones, bipyridylium salts, carbamates, chloroacetamides, chlorocarboxylic acids, cyclohexanediones, dinitroanilines, dinitrophenols, diphenyl ethers, glycines, imidazolinones, isoxazoles, isoxazolidinones, nitriles, N-phenylphthalimides, oxadiazoles, oxazolidinediones, oxyacetamides, phenoxycarboxylic acids, phenyl carbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl (thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas and the agriculturally acceptable salts and derivatives thereof.

In a specific subgroup of this embodiment, the crop protecting agent is a crop protecting agent which is liquid at 22° C. and 1013 mbar or a mixture of two or more crop protecting agents which is liquid at 22° C. and 1013 mbar. Examples of room temperature liquid active ingredients are dimethenamid, especially the enantiomer thereof dimethenamid-P, clomazone, metolachlor, especially the enantiomer thereof S-metolachlor. In a further specific subgroup of this embodiment, the crop protecting agent is a crop protecting agent with low water solubility and a melting point of not more than 110° C. or a mixture of such active ingredients. These include, for example, pyrachlostrobin (64° C.), prochloraz (47° C.), metrafenon (100° C.), alphacypermethrin (79° C.) and pendimethalin (58° C.).

In a further group of embodiments, the organic active of low molecular weight is an organic active suitable for cosmetic applications or an active mixture other than the aforementioned aromas. Preferred cosmetic actives for loading of the microparticles are especially active plant ingredients and plant extracts.

Examples of cosmetic actives are skin and hair pigmentation agents, tanning agents, bleaches, keratin-hardening substances, antimicrobial active ingredients, photofilter active ingredients, repellent active ingredients, hyperemic substances, keratolytic and keratoplastic substances, antidandruff active ingredients, antiphlogistics, keratinizing substances, antioxidative active ingredients and active ingredients acting as free-radical scavengers, skin moisturizing or humectant substances, regreasing active ingredients, deodorizing active ingredients, sebostatic active ingredients, plant extracts, antierythematous or antiallergic active ingredients and mixtures thereof.

Artificial tanning actives suitable for tanning the skin without natural or artificial irradiation with UV rays are, for example, dihydroxyacetone, alloxan and walnut shell extract. Suitable keratin-hardening substances are generally active ingredients as are also used in antiperspirants, for example potassium aluminum sulfate, aluminum hydroxychloride, aluminum lactate, etc. Antimicrobial active ingredients are used in order to destroy microorganisms and/or to inhibit their growth and thus serve both as preservatives and also as a deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid, etc. Such deodorizing substances are, for example, zinc ricinoleate, triclosan, undecylenoic acid alkylolamides, triethyl citrate, chlorhexidine, etc. Suitable photofilter active ingredients are substances which absorb UV rays in the UV-B and/or UV-A region. Suitable UV filters are those mentioned above. Additionally suitable are p-aminobenzoic esters, cinnamic esters, benzophenones, camphor derivatives, and pigments which stop UV rays, such as titanium dioxide, talc and zinc oxide. Suitable repellent active ingredients are compounds capable of warding off or driving away certain animals, particularly insects, from humans. These include, for example, 2-ethyl-1,3-hexanediol, N,N-diethyl-m-toluamide, etc. Suitable hyperemic substances, which stimulate blood flow through the skin, are, for example, essential oils, such as dwarf pine, lavender, rosemary, juniperberry, roast chestnut extract, birch leaf extract, hayseed extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counter skin irritations, are, for example, allantoin, bisabolol, Dragosantol, chamomile extract, panthenol, etc.

Further cosmetic actives are aspalatin, glycyrrhizin, caffeine, proanthocyanidin, hesperetin, rutin, luteolin, polyphenols, oleuropein, theobromine, bioflavonoids and polyphenols.

Examples of plant extracts are also acai extract (Euterpe oleracea), acerola extract (Malpighia glabra), field horsetail extract (Equisetum arvense), agarius extract (Agarius blazei murill), aloe extract (Aloe vera, Aloe Barbadensis), apple extract (Malus), artichoke leaf extract (Cynara scolymus), artichoke blossom extract (Cynara edulis), arnica extract (Arnica Montana), oyster extract (Ostrea edulis), baldrian root extract (Valeriana officinalis), bearberry leaf extract (Arctostaphylos uva-ursi), bamboo extract (Bambus vulgaris), bitter melon extract (Momordica charantia), bitter orange extract (Citrus aurantium), nettle leaf extract (Urtica dioica), nettle root extract (Urtica dioica), broccoli extract (Brassica oleracea), watercress extract (Rorippa nasturtium), painted nettle extract (Coleus forskohlii), capsicum extract (Capsicum frutescens), extract from Centella asiatica (Gotu Kola), cinchona extract, cranberry extract (Vaccinium vitisdaea), turmeric extract (Curcuma longa), damiana extract (Tunera diffusa), dragonfruit extract (Pitahaya), extract from Echinacea purpurea, wheat placenta extract, edelweiss extract (Leotopodium alpinum), ivy extract (Hedera helix), bindii extract (Tribulus terrestris), Garcinia cambogia extract (Garcinia cambogia), ginkgo extract (Ginkgo biloba), ginseng extract (Panax ginseng), pomegranate extract (Punica granatum), grapefruit extract (Citrus paradisi), griffonia extract (Griffonia simplicifolia), green tea extract (Camellia sinensis), guarana extract (Paullinia cupana), cucumber extract (Cucumis sativus), dog rose extract (Rosa canina), blueberry extract (Vaccinium myrtillus), hibiscus extract (Malvacea), mallow extract, honey extracts, hops extract (Humulus), ginger extract (Zingiber officinale), Iceland moss extract (Cetraria islandica), jojoba extract (Simmondsia chinensis), St. John's Wort extract (Hypericum perforatum), coffee concentrate, cocoa bean extract (Theobroma cacao), cactus blossom extract, chamomile blossom extract (Matricaria recutita, Matricaria chamomile), carrot extract (Daucus carota), kiwi extract (Aperygidae), kudzu extract (Pueraria lobata), coconut milk extract, pumpkinseed extract (Curcurbita pepo), cornflower extract (Centaurea cyanus), lotus flower extract, dandelion extract (Taraxacum officinale), maca extract (Lepidium peruvianum), magnolia blossom extract, mango extracts, milk thistle extract (Silybum marianum), marigold extract (Calendula officiennalis), mate extract (Hex paraguariensis), butcher's broom extract (Rugcus aculeatus), sea algae extracts, cranberry extracts (Vaccinium macrocarpon), Moringa Oleifera extract, extract from Moschus Malve (Malva moschata), evening primrose oil extract (Azadirachta indica), nettle extract (Urticaceae), olive leaf extract (Olea europea), orange extract (hesperidin), orchid extract, papaya extract (Carica papaya), peppermint extracts, extract from Carica papaya (Geissospermum), bitter orange extract (Citrus aurantioum), lingonberry extract (Vaccinium vitas-ideea), African cherry extract (Prunus africana), sugar beet extracts, resveratrole extract (Polygonum cuspidatum), rooibos extract (Aspalasthus Linnearis), rose blossom extract, horse chestnut extract (Aesculus hippocastanum), rosemary extract (Rosemarinus Officinalis), red clover extract (Trifolium platense), red wine extract (Vitis vinifera), saw palmetto extract (Serenoa repens), lettuce extract (Lactuca sativa), sandalwood extract (Santalum rubrum), sage extract (Salvia officinalis), horsetail extract (Equisetum), yarrow extract (Achillea millefolium), black pepper extract (Piper nigrum), black tea extract, waterlily extract (Nymphaea), white willow bark extract (Salix Alba), liquorice extract (Glycyrrhiza), devil's claw extract (Harpagophytum procumbens), thyme extract (Thymus vulgaris), tomato extract (Lycopersicum esculentum), grapeseed extract (Vitis vinifera), grapeskin extract (Vitis vinifera), watercress (Rorippa amphibia), willow bark extract (Salix alba), wormwood extract (Artemisia absinthium), white tea extract, yam root extract (Dioscorea opposita), yohimbe extract (Pausinystalia yohimbe), witch hazel extract (Hamamelis), cinnamon extract (Cinnamomum cassia Presl), lemon extract (Citrus) and onion extract (Allium cepa).

In a further group of embodiments, the organic active of low molecular weight is an organic active from construction chemistry. Preferred actives for loading of the microparticles for applications in construction chemistry are especially polymerization catalysts.

Useful polymerization catalysts include those suitable for curing of reactive resins, especially addition resins, condensation resins or oxidation-curing resins. For this purpose, the polymerization catalyst is a catalyst for a free-radical polymerization, a polycondensation and/or a polyaddition. The suitable catalysts for a free-radical polymerization especially include peroxide splitters, and the catalysts known from coatings technology for oxidatively drying oil and alkyd resins as driers or siccatives. Suitable polycondensation catalysts are catalysts for silicone condensation and crosslinking. Polyaddition catalysts used may, for example, be catalysts for curing of epoxy resins. In addition, polyaddition catalysts used may, for example, be urethanization catalysts customarily used in polyurethane chemistry. These are compounds that accelerate the reaction of the reactive hydrogen atoms of isocyanate-reactive components with the organic polyisocyanates.

Useful polymerization catalysts especially include amines, phosphines and organic metal salts.

Amines useful as polymerization catalysts, especially for polyadditions, are especially tertiary amines such as triethylamine, tributylamine, N,N-dimethylcyclohexylamine (DMCHA), N-methyldicyclohexylamine, N,N-dimethylbenzylamine (BDMA), N-methylmorpholine, N-ethylmorpholine, N-cyclohexylmorpholine, 2,2′-dimorpholinodiethyl ether (DMDEE), N,N,N′,N′-tetramethylethylenediamine, N,N,N′,N′-tetramethylbutylene-diamine, N,N,N′,N′-tetramethylhexylene-1,6-diamine, N,N,N′,N″,N″-pentamethyldiethylenetriamine (PMDETA), N,N,N′,N″,N″-pentamethyldipropylenetriamine (PMDPTA), N,N,N-tris(3-dimethylaminopropyl)amine, bis(2-dimethylaminoethyl) ether (BDMAEE), bis(dimethylaminopropyl)urea, 2,4,6-tris(dimethylaminomethyl)phenol, and its salt with 2-ethylhexanoic acid and isomers thereof, 1,4-dimethylpiperazine (DMP), N-methylimidazole, 1,2-dimethylimidazole, 1-methyl-4-(2-dimethylaminoethyl) piperazine, 1-azabicyclo[3.3.0]octane, 1,4-diazabicyclo[2.2.2]octane (DABCO), 1,8-diazabicyclo[5.4.0]undec-7-ene (DBU), 1,5-diazabicyclo[4.3.0]non-7-ene (DBN).

Further useful polymerization catalysts, especially for polyadditions, include: tris(dialkylamino)-s-hexahydrotriazines, especially 1,3,5-tris(3-[dimethylamino]propyl) hexahydrotriazine.

It is also possible to use polymerization catalysts that are reactive towards isocyanates, especially for polyadditions. Besides at least one tertiary amino group, they comprise a primary or secondary amino group or a hydroxyl group. Examples thereof include N,N-dimethylaminopropylamine, bis(dimethylaminopropyl)amine, N,N-dimethylaminopropyl-N′-methylethanolamine, dimethylaminoethoxyethanol, bis(dimethylaminopropyl)amino-2-propanol, N,N-dimethylaminopropyldipropanolamine, N,N,N′-trimethyl-N′-hydroxyethyl bisaminoethyl ether, N,N-dimethylaminopropylurea, N-(2-hydroxypropyl)imidazole, N-(2-hydroxyethyl)imidazole, N-(2-aminopropyl)imidazole and/or the reaction products of ethyl acetoacetate, polyether polyols and 1-(dimethylamino)-3-aminopropane that are described in EP-A 0 629 607.

Useful phosphines as polymerization catalysts, especially for polyadditions, are preferably tertiary phosphines, such as triphenylphosphine or methyldiphenylphosphine.

Organic metal salts useful as polymerization catalysts preferably have the general formula

LₘMⁿ⁺nA⁻

in which

the ligand L is an organic radical or an organic compound selected from alkyl, alkenyl, alkynyl, aryl, arylalkyl, alkylaryl, heteroaryl, heteroarylalkyl, alkylheteroaryl and acyl, the ligand L having 1 to 20 carbon atoms, and the m ligands L being the same or different,

m is 0, 1, 2, 3, 4, 5 or 6,

M is a metal,

n is 1, 2, 3 or 4, and

the anion A⁻ is a carboxylate ion, alkoxylate ion or enolate ion.

The metal M is preferably selected from lithium, potassium, cesium, magnesium, calcium, strontium, barium, boron, aluminum, indium, tin, lead, bismuth, cerium, cobalt, iron, copper, lanthanum, manganese, mercury, scandium, titanium, zinc and zirconium; more particularly from lithium, potassium, cesium, tin, bismuth, titanium, zinc and zirconium.

The ligand L is preferably alkyl having 1 to 20 carbon atoms. More preferably L is alkyl having 1 to 10 carbon atoms, especially 1 to 4 carbon atoms, e.g. methyl, ethyl, n-propyl, isopropyl, n-butyl, isobutyl, sec-butyl or tert-butyl.

The carboxylate ion preferably has the formula R¹—COO⁻ where R¹ is selected from H, alkyl, alkenyl, alkynyl, aryl, arylalkyl, alkylaryl, heteroaryl, heteroarylalkyl, alkylheteroaryl and acyl, and where the R¹ radical has up to 20 carbon atoms, preferably 6 to 20 carbon atoms. Particularly preferred carboxylate ions are selected from the anions of natural and synthetic fatty acids, such as neodecanoate, isooctanoate and laurate, and the anions of resin acids and naphthenic acids.

The enolate ion preferably has the formula R²CH═CR³—O⁻ where R² and R³ are each selected from H, alkyl, alkenyl, alkynyl, aryl, arylalkyl, alkylaryl, heteroaryl, heteroarylalkyl, alkylheteroaryl and acyl, and where the R² and R³ radicals each have up to 20 carbon atoms. Specific examples are ethylacetonate, heptylacetonate or phenylacetonate. The enolate ion derives preferably from a 1,3-diketone having five to eight carbon atoms. Possible examples include acetylacetonate, the enolate of 2,4-hexanedione, the enolate of 3,5-heptanedione and the enolate of 3,5-octanedione.

The alkoxylate ion preferably has the formula R⁴—O⁻ where R⁴ is selected from alkyl, alkenyl, alkynyl, aryl, arylalkyl, alkylaryl, heteroaryl, heteroarylalkyl, alkylheteroaryl and acyl, and where the R⁴ radical has up to 20 carbon atoms.

In particular embodiments the organic metal compound is selected from
alkali metal carboxylates, such as lithium ethylhexanoate, lithium neodecanoate, potassium acetate, potassium ethylhexanoate, cesium ethylhexanoate;
alkaline earth metal carboxylates, such as calcium ethylhexanoate, calcium naphthenate, calcium octoate (available as Octa-Soligen® Calcium from OMG Borchers), magnesium stearate, strontium ethylhexanoate, barium ethylhexanoate, barium naphthenate, barium neodecanoate;
aluminum compounds, such as aluminum acetylacetonate, aluminum dionate (e.g. K KAT® 5218 from King Industries);
zinc compounds, for example zinc(II) diacetate, zinc(II) ethylhexanoate and zinc(II) octoate, zinc neodecanoate, zinc acetylacetonate;
tin compounds, such as tin(II) carboxylates, examples being tin(II) acetate, tin(II) octoate, tin(II) ethylhexanoate, tin(II) neodecanoate, tin(II) isononanoate, tin(II) laurate, and dialkyltin(IV) salts of organic carboxylic acids, examples being dimethyltin diacetate, dibutyltin diacetate, dibutyltin dibutyrate, dibutyltin bis(2-ethylhexanoate), dibutyltin dilaurate, dibutyltin maleate, dioctyltin dilaurate and dioctyltin diacetate, especially dibutyltin dilaurate;
titanium compounds, such as tetra(2-ethylhexyl) titanate;
zirconium compounds, such as zirconium ethylhexanoate, zirconium neodecanoate, zirconium acetylacetonate (e.g. K-KAT® 4205 from King Industries); zirconium dionates (e.g. K-KAT® XC-9213; XC-A 209 and XC-6212 from King Industries); zirconium 2,2,6,6-tetramethyl-3,5-heptanedionate;
bismuth compounds, such as bismuth carboxylates, especially bismuth octoate, bismuth ethylhexanoate, bismuth neodecanoate or bismuth pivalate (e.g. K-KAT® 348, XC-B221, XC-C227, XC 8203 from King Industries, TIB KAT 716, 716LA, 716XLA, 718, 720, 789 from TIB Chemicals, and those from Shepherd Lausanne);
manganese salts, such as manganese neodecanoate, manganese naphthenate;
cobalt salts, such as cobalt neodecanoate, cobalt ethylhexanoate, cobalt naphthenate;
iron salts, such as iron ethylhexanoate;
mercury compounds, such as phenylmercury carboxylate.

Preferred organic metal compounds are dibutyltin dilaurate, dioctyltin dilaurate, zinc(II) diacetate, zinc(II) dioctoate, zirconium acetylacetonate and zirconium 2,2,6,6-tetramethyl-3,5-heptanedionate, bismuth neodecanoate, bismuth dioctoate and bismuth ethylhexanoate.

According to step (a) of the process of the invention a composition of unladen microparticles are impregnated with a liquid comprising the active, whereby laden microparticels are obtained, which in their interior cavity contain the liquid and, hence, the active. For this, the microparticles are treated with the liquid treated in such a way that the cavity present in the unfilled microparticles is largely filled by the liquid, i.e. at least to an extent of 50%, especially at least to an extent of 70%, or completely, or the majority of the gas present in the microparticles is displaced by the liquid. The liquids comprise the active, and so, when the microparticles are treated with the liquid, the active penetrates through the pores into the cavities of the microparticles and hence into the interior of the microparticles.

For impregnation of the microparticles with the liquid the microparticles to be laden will generally be contacted with the liquid containing the respective active. The contacting can in principle be effected in any desired manner, with the proviso that the contact time is sufficient that the liquid has at least wetted the microcapsules to be loaded and hence can penetrate via the pores into the cavities of the microparticles. For this purpose, the microparticles to be laden and the respective liquid are typically mixed with one another, for example by applying the respective liquid containing the active to the microparticles to be laden in finely distributed form, e.g. as droplets, or by suspending the microparticles in the respective liquid.

According to a group of embodiments, the unloaded microparticles are impregnated by a method, which comprises suspending the microparticles in a liquid, which contains the active desired to be laden.

According to another group of embodiments, the microparticles are impregnated by a method, which comprises applying the liquid containing the active to the unloaded microparticles in finely distributed form, in particular as droplets. For this purpose, the microparticles will usually be used in solid form, in particular in the form of a powder. In particular, the unloaded microparticles can be sprayed or dripped as powder with the respective liquid containing the active substance. Surprisingly, the liquid droplets are rapidly absorbed by the unloaded microparticles. In addition, the liquid used for impregnation and thus the active can be precisely dosed in this way so that separation of excess liquid can be avoided or the associated expense can be reduced.

The unloaded microparticles are typically impregnated with the respective liquid containing the active at temperatures below the melting or softening point or range of the wall material, which can be determined by the person skilled in the art in a manner known per se, for example by means of differential scanning calorimetry (DSC). Typically, the treatment is effected at temperatures that are at least 5 K, especially at least 10 K, below the melting or softening point or range of the wall material, for example at not more than 80° C., particularly not more than 70° C., especially not more than 60° C., for example in the range from 0 to 80° C., particularly in the range from 10 to 70° C. and especially in the range from 20 to 60° C. The treatment time is typically in the range from 1 min to 10 h, particularly in the range from 5 min to 8 h and especially in the range from 0.5 to 6 h.

The active may be in liquid or molten form in the liquids or in liquid or molten form in the simultaneous presence of a solvent, in emulsified or suspended form, or preferably in dissolved form. The active may also be present as such provided that the active is liquid at the temperature at which the impregnation is carried out, for example in the case of an active or active mixture that is liquid at room temperature (22° C.).

If the active is in liquid or molten form in the liquid, it may have entirely or partly melted. Preferably, the active has entirely melted especially has a temperature of more than 5° C. above its melting point in the loading. If the active is in emulsified form in the liquid, it may either be the disperse phase or the continuous phase. If the active is in suspended form, it preferably has a particle size smaller than the average pore radius.

If the active is in dissolved form, it may have entirely or partly dissolved. The active has preferably entirely dissolved. The dissolution of the active can in principle be brought about by any components of the liquid in which the active is soluble, for example a solvent or solvent mixture or an optional further active.

Alternatively, it is possible to use organic solvents or water or mixtures of water and water-miscible organic solvents. Suitable organic solvents are especially those in which the wall material is insoluble. Suitable organic solvents are especially C₁-C₄-alkanols such as methanol, ethanol, n-propanol, isopropanol, n-butanol, 2-butanol, tert-butanol and aliphatic and cycloaliphatic hydrocarbons such as n-pentane, n-hexane, hexane mixtures, n-heptane, cyclohexane, cycloheptane, methylcyclohexane, petroleum ether, white oils, diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-alkyl esters of aliphatic, aromatic or cycloaliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl cyclohexane-1,2-dicarboxylate, and dialkyl adipates such as dibutyl adipate (e.g. Cetiol® B from BASF SE), C₈-C₂₂ fatty acid triglycerides, e.g. vegetable oils or cosmetic oils such as octanoyl/decanoyltriglyceride (e.g. the commercial product Myritol® 318 from BASF SE), and dimethyl sulfoxide and mixtures thereof.

The microparticles to be laden with the active may be used in the form of a particulate solid, e.g. a powder, or as a suspension in a solvent in which the wall material is insoluble. Preference is given to using the microparticles to be treated in the form of a powder. This can be suspended in the liquid containing the respective active and is preferably applied dropwise or by spray application with the liquid. Alternatively, it is possible to mix a suspension of the microparticles to be laden with the liquid containing the active.

Examples of suitable apparatus for production of the suspension include magnetic stirrers, rollers, shakers, and various close-clearance stirrer units, e.g. anchor stirrers, helical stirrers. The duration of the mixing operation depends on the viscosity of the liquid at loading temperature and hence on the diffusion rate of the liquid into the microparticles, and is generally 5 minutes to 12 hours. Preferably, 1 part by weight of microparticles is suspended in 0.5 to 5 parts by weight, preferably 0.8 to 3 parts by weight, of the respective liquid. The suspension comprising the microparticles and the respective liquid is generally kept at a temperature in the range from 0 to 80° C. for 1 minute to 10 hours. The suspension is preferably kept at a temperature in the range from 10 to 70° C. and especially in the range from 20 to 60° C. for 0.5 hour to 10 hours. Optionally, the microparticles are separated from the liquid added in excess. The methods suitable for this purpose are, for example, filtration, centrifugation, decanting and drying, for example contact drying, fluidized bed drying, vacuum drying or spray drying.

Preferably, the unloaded microparticles are impregnated with a liquid containing the active ingredient by a method other than suspending the unloaded microparticles in a liquid containing the active ingredient. In particular, in order to impregnate the unladen microparticles, a liquid containing the active substance will be applied in finely divided form, in particular in the form of droplets, to the unloaded microparticles. The microparticles are usually applied in solid form, especially in the form of powder. In particular, a powder of the unloaded microparticles will be dripped or sprayed with the respective liquid containing the active substance.

In general, for this purpose, the unloaded microparticles will be initially charged in a mixer for the mixing of solids with liquids and the liquid containing the active will be added, preferably in finely divided form, for example in the form of large droplets or as a spray mist of fine droplets. In particular, the liquid will be applied to the laden microparticles that are in motion.

For example, the microparticles to be loaded can be moved in a suitable manner, in particular a fluidized bed of the microparticles to be loaded can be produced, and the liquid containing the active substance can be applied in finely distributed form to the moving microparticles or microparticles in the fluidized bed, e.g. by spraying or dropping. Spraying or dripping can be carried out in a manner known per se by means of one or more nozzles, e.g. by means of single-substance or two-substance nozzles or by means of dripping devices. Suitable mixing devices are dynamic mixers, in particular compulsory mixers, or mixers with a mixer shaft, e.g. shovel mixers, paddle mixers or ploughshare mixers, but also free-fall mixers of this kind, e.g. drum mixers, and fluidized bed mixers. The duration of the mixing operation depends on the type of mixer and the viscosity of the liquid at loading temperature and hence on the diffusion rate of the liquid into the microparticles. The time required for loading can be determined in a simple manner by the person skilled in the art. It is generally 1 minute to 5 hours, in particular 2 minutes to 2 hours or 3 minutes to 1 hour. Preferably, the liquid containing the respective active is used in an amount of 0.2 to 5 parts by weight, preferably 0.5 to 4 parts by weight, based on 1 part by weight of the unladen microparticles. The spray application or dropwise application is generally at a temperature in the range from 0 to 80° C., particularly in the range from 10 to 70° C. and especially in the range from 20 to 60° C.

It may be advantageous to remove any residual water present from the laden microparticles. This can be effected, for example, by rinsing with ethanol or acetone, and/or blowing the microparticles dry with an inert gas such as air, nitrogen or argon. Optionally, for this purpose, predried and/or preheated inert gases may also be used. Preferably, the filled microparticles are subsequently washed, preferably with aqueous propanediol solution, for example as a 10% by weight solution.

Commonly known drying methods may be used for drying. For example, the laden microparticles may be dried by means of convective driers such as spray driers, fluidized bed driers, cyclone driers, contact driers such as pan driers, contact belt driers, vacuum drying cabinet or radiative driers such as infrared rotary tube driers and microwave mixing driers.

Thereby laden microparticles, in particular spherical microparticles are obtained which contain the desired active and which subjected are subjected to thermal treatment of step b) of the process of the invention. The amount of active in laden microparticles depends from the volume of the cavities in the interior of the unloaded microparticles and is usually in the range from 5 to 75% by weight, in particular in the range from 10 to 60% by weight, based on the total weight of the laden microparticles.

As the loading of step a) does not significantly alter the particle size distribution as well as the amount and sizes of the pores it is apparent that the laden microparticles which are subjected to the thermal treatment of step b) have particle sizes (i.e. D[4,3], D[3,2], D[v, 0.1], D[v, 0.5] and D[v, 0.9] values), amounts of pores and pore sizes which are similar to the of the unloaded microparticles employed in step a). Preferably, the laden microparticles have an average particle diameter D[4,3] of 5 to 600 μm, particularly of 7 to 500 μm and especially in the range of 10 to 400 μm. Preferably, the laden microparticles have at least 10, preferably at least 20, pores on their surface. Preferably, the diameter of the pores is in the range from 20 nm to 1/5 of the average particle diameter. The diameter of these pores is preferably at least 50 nm, more preferably at least 100 nm and especially at least 200 nm. The diameter of these pores will generally not exceed 20 μm, especially 10 μm, and is preferably in the range of 50 nm to 20 μm, in particular in the range of 100 nm to 20 μm and especially in the range of 200 nm to 10 μm, depending on the respective average particle diameter D[4,3].

In step b) of the claimed process, the laden microparticles obtained in step a) are subjected to thermal treatment by passing a stream of free flowing laden microparticles in a carrier gas through a heated zone. In other words, the laden microparticles are passed through the heated zone as a stream of free flowing laden microparticles in a carrier gas.

By the thermal treatment of step b) the pores of the laden microparticles are closed. It is believed that the elevated temperatures in the heated zone the wall material of the laden microparticles softens in particular at the rims of the pore openings, which leads to a contraction and closure of the pore openings due to capillary forces and surface tension, respectively. By passing the free flowing stream of microparticles in a carrier gas through the heated zone, a homogeneous and isolated distribution of the microparticles is achieved and thereby agglomeration of the hot microparticles in the heated zone can be reduced or avoided. Thereby a rapid and uniform, hence, efficient closure of the pores is achieved without significant increase in particle size resulting from unwanted agglomeration.

The temperature of the heated zone is at least 20 K, in particular at least 30 K and especially at least 40 K above the softening temperature of the thermoplastic organic polymeric material of the microparticles, and frequently in the range of 20 to 300 K or in the range of 20 to 250 K or in the range of 20 to 220 K or in the range of 20 to 220 K, in particular in the range of 30 to 250 K or in the range of 30 to 220 K or in the range of 30 to 200 K and especially in the range of 40 to 250 K or in the range of 40 to 220 K or in the range of 40 to 200 K or in the range of 40 to 180 K above the softening temperature of the thermoplastic organic polymeric material of the microparticles. The values given herein refer to the Vicat softening temperature, especially to the VST/A50 of the thermoplastic organic polymeric material which forms the wall material of the unloaded microparticles.

Frequently, the temperature in the heated zone is in the range of 130 to 350° C., in particular in the range of 140 to 300° C., especially in the range of 140 to 250° C. The heated zone may have an almost uniform or homogeneous temperature profile, i.e. the temperatures at to different points in the heated zone do not differ by more than 10° C. Frequently, the temperature at the inlet of the heated zone may be higher than the temperature at the outlet.

The temperature in the heating zone can be set by heating the walls of the heating zone with an external heater, e.g. by an electric heater or by hot steam, or with infrared radiators, or with a gas flow heated to the desired temperature, or by a combination of these measures.

Due to the high temperatures, the time required for efficient closure of the pore openings is quite short and average residence times of the laden particles in the heated zone of not more than 60 seconds in particular of not more than 30 seconds are sufficient for achieving a complete or at least virtually complete closure of the pore openings. Therefore, the average residence time of the laden microparticles in the heated zone will not exceed 60 s, in particular 30 s, more particularly 20 s and especially 10 s and is e.g. in the range of 0.1 to 60 s, in particular in the range of 0.1 to 30 s, more particularly in the range of 0.5 to 20 s and especially in the range of 1 to 10 s.

It is apparent that residence time for achieving an efficient, i.e. a virtually complete closure of the pores of the microparticles will reciprocally depend from the temperature of the heated zone. The same degree of closure can be achieved, for example, with a short residence time of the microparticles and a high temperature, while longer residence times are required at lower temperatures. In case of volatile or heat-sensitive actives it short residence times and higher temperatures may be preferred rather than long residence times and lower temperatures. Of course, the optimum conditions for efficient closure of the pores will also depend from the type of wall material. If the wall material has a high melting point/glass transition temperature or contains a large proportion of amorphous material, higher temperatures and/or longer residence time will be required while a high degree of crystallinity and/or low melting points allow for shorter residence times and/or lower temperatures. A skilled person will find optimum conditions for achieving efficient closure and minimizing loss of active by routine.

By choosing a proper mass density of the laden microparticles in the free flowing gas stream, agglomeration caused by mutual contact of the laden microparticles in the free flowing gas stream can be minimized. The mass density of the stream of free flowing laden microparticles passed through the heated zone will preferably not exceed 500 g/m³, in particular 200 g/m³ and especially 100 g/m³. The mass density of the stream of free flowing laden microparticles frequently in the range from 5 to 500 g/m³, in particular in the range from 5 to 200 g/m³ and especially in the range from 10 to 100 g/m³.

The carrier gas can be any gas that is chemically inert or almost inert to the laden microparticles at the temperature of the heated zone. Usually air is sufficiently inert to the laden microparticles at the temperature given above and thus may be used as carrier gas. However, in case of a sensitive active an inert gas such as nitrogen or argon may be used instead of air or the partial pressure of oxygen in the air may be reduced by using a mixture of air and nitrogen.

In order to minimize the contact of the microparticles with the hot walls of the heated zone, it is preferred that the stream of free flowing laden microparticles passed is through the heated zone such that the stream exhibits an essentially laminar flow when it is passed through the heated zone. The flow characteristics will depend on the flow velocity of the stream of microparticles and the geometry of the heated zone. A skilled person will find by routine proper flow velocities for a given geometry of the heated zone, which result in an essentially laminar flow of free flowing laden microparticles passed through the heated zone.

In order to achieve a stream of free flowing laden microparticles in a carrier gas, the laden microparticles are usually mixed with the carrier gas before the stream enters the heated zone. The mixing may be carried out in a separate step, for example in the drying step, if a fluidized bed dryer or a similar equipment is used for drying. The mixing may also be carried out immediately before the carrier gas enters into the heated zone. For example, the microparticles may be dosed into the stream of a carrier gas by means of a suitable dosing equipment. Such a dosing equipment is usually designed to achieve a homogeneous and isolated distribution of the microparticles in the stream of the carrier gas in order to prevent agglomeration of the particles in the heating zone and to achieve as uniform a closure of the particles as possible. Suitable dosing equipment include powder injection lances, pneumatic conveyers and dosing screws. It is also possible to separate a gas stream containing laden microparticles from a drying equipment, e.g. a fluidized bed dryer or a similar equipment, and then mix this stream with further carrier gas to adjust the desired mass density. Usually, a powder of the laden microparticles will be mixed with the carrier gas but it is also possible to mix a suspension of the microparticles in a volatile liquid, such as water, with the carrier gas.

In general, the geometry of the heated zone is such that wall contact of the microparticles with the hot inner walls of the heated zone is minimized or avoided, when the stream of the carrier gas containing the free flowing microparticles is passed through the heated zone.

Preferably, the heated zone has an oblong shape, which means that the length of the spatial cure, i.e. the distance between the inlet and the outlet of the heated zone along the spatial curve perpendicular to all cross-sections of the heated zone, is significantly longer than the average cross-section of the heated zone. In particular ratio of the length of the spatial curve to the average cross-section is at least 2, in particular at least 5 and may be as high as 100 or higher and is in particular in the range from 5 to 100 preferably in the range from 7 to 50 and especially in the range from 10 to 30.

Preferably, the heated zone has an essentially straight geometry, i.e. the angular deviation of the spatial curve through the inlet and the outlet of the heated zone from a line perpendicular to the cross-sections of the heated zone at the inlet and outlet will not exceed 20° m⁻¹, in particular 10° m⁻¹, and is preferably 0° m⁻¹, or close to 0° m⁻¹.

In case of a heated zone having a straight geometry, the heated zone is preferably arranged perpendicular, i.e. the spatial curve through the inlet and the outlet of the heated zone is preferably parallel or close to parallel to the gravitational field.

Preferably, the heated zone has a tubular geometry. The cross-sectional area of the heated tube can have any shape. In one embodiment, the cross sectional area of the heated zone is polygonal area with 4 corners, e.g. a rectangular area such that the heated zone is defined by rectangular tube. In another embodiment, the cross-sectional area is polygonal area with at least 6 corners, or ellipsoidal or circular area. In particular the cross section area of the heated zone is elliptic or especially circular.

Preferably, the internal surface of the heated zone has essentially no narrow edges with angles of less than 90°.

In particular, the heated zone has a straight tubular geometry, i.e. it has a straight oblong shape with elliptic or circular cross-section areas. In case of elliptic cross-section areas the ratio of the longest axis to the shortest axis will preferably not exceed 4:1. In particular, the cross-sectional area of such a heated zone perpendicular to the spatial curve through the inlet and the outlet of the heated zone is ellipsoid or circular.

However, it is also possible that the heated zone has straight channel-type geometry, i.e. it has a straight oblong shape with rectangular or higher polygonal cross-sections and corner angles of ≥90°. In case the cross-section areas of heated zone are rectangular or polygonal the ratio of the longest axis to the shortest axis will preferably not exceed 4:1.

In a particularly preferred group of embodiments, the heated zone is arranged in a downpipe, i.e. the heated zone has a straight tubular geometry, which is arranged perpendicular and where the stream of free flowing laden microparticles is passed downwards, i.e. from the upper end to the lower end of the heated zone.

In another particularly preferred group of embodiments, the heated zone is arranged in a raiser pipe, i.e. the heated zone has a straight tubular geometry, which is arranged perpendicular and where the stream of free flowing laden microparticles is passed upwards, i.e. from the upper end to the upper end of the heated zone.

It may be beneficial to quench the hot microparticles immediately after they leave the heated zone, in order to solidify the melted surface again and prevent agglomeration. The term “quench” is understood to rapidly cool the hot microparticles to a temperature below the softening temperature of the wall material, in particular to a temperature, which is at least 10 K below the softening temperature of the wall material. Quenching may be achieved by mixing a cold gas stream with the hot stream of microparticles leaving the heated zone, in particular by injecting a cold gas stream with the hot stream of microparticles leaving the heated zone. Quenching may also be achieved by introducing the hot stream of microparticles leaving the heated zone into a cold fluidized bed of microparticles having already been subjected to step b) or by mixing the hot stream hot stream of microparticles leaving the heated zone with a cold liquid. Alternatively or in combination with the quenching an axial temperature profile of the heated zone may be applied, where the axial temperature profile has a temperature at the outlet of the heated zone, which is significantly lower than the softening point of the wall material. In particular, such an axial temperature profile will have a temperature at the inlet of the heated zone of more than 20 K above the softening point of the wall material and a temperature material at the outlet of the heated zone of at least 10 K below the softening point of the wall.

The thermal treatment of step b) results in microparticles, in particular spherical microparticles, which contain the active captured in the interior cavity/cavities of the microparticles. The pores present in the microparticles before step b) is carried out are efficiently closed, i.e. the average number of pores still present after step b) was carried out is less than 20%, in particular less than 10% of the number of pores present in the unloaded microparticles subjected to step a). Likewise the average area of pore openings in the surface of the microparticles is efficiently reduced by at least 60%, in particular at least 80% of the average area of pore openings in the surface of the unloaded microparticles subjected to step a).

The amount of active in microparticles after step b) has been carried out is usually at least 50%, in particular at least 80% of the amount of active in microparticles before step b) is carried out. The amount of active in microparticles after step b) is usually in the range from 2 to 70% by weight, in particular in the range from 4 to 55% by weight, based on the total weight of the microparticles obtained in step b).

As step b) does not significantly alter the particle size distribution, the microparticles obtainable from the thermal treatment of step b) have particle sizes (i.e. D[4,3], D[3,2], D[v, 0.1], D[v, 0.5] and D[v, 0.9] values), which are similar to the of the unloaded microparticles employed in step a). Typically, the microparticles obtainable from step b) have an average particle diameter D[4,3] in the range of 5 to 600 μm, particularly in the range of 7 to 500 μm and especially in the range of 10 to 400 μm. Typically, the D[v, 0.9] value of the particle size distribution of the microparticles obtainable from step b) does not exceed 1200 μm, particularly 900 μm and especially 700 μm. Typically, the D[v, 0.1] value of the particle size distribution of the microparticles obtainable from step b) is at least 1 μm, particularly at least 2 μm and especially at least 5 μm. Typically, the D[v, 0.5] value of the particle size distribution of the microparticles obtainable from step b) is in the range of 3 to 500 μm, particularly in the range of 4 to 300 μm and especially in the range of 8 to 300 μm. Typically, the microparticles obtainable from step b) have an D[3,2] value in the range of 2.5 to 400 μm, particularly in the range of 3.5 to 250 μm and especially in the range of 5 to 200 μm.

The present invention further provides compositions of the microparticles filled with at least one active, obtainable by the process of the invention. The compositions of the invention preferably obtain the active in a total amount of 2 to 70% by weight, in particular 5 to 60% by weight, based on the total weight of the microparticles laden with the actives, i.e. of the constituents of the composition other than solvents. The constituents of the microparticles, i.e. the constituents of the composition other than solvents, are essentially the active and the polymer that forms the wall material and any auxiliaries that are used in the production of the microparticles or in the loading of the microparticles and are not removed. The compositions of the invention may be in the form either of a suspension or of a powder, preference being given to powders.

The present invention further provides products comprising a composition of the invention. Preference is given to products comprising the compositions of the invention in a proportion by weight of 0.01% to 80% by weight, based on the total weight of the product.

The nature of the product is naturally guided by the nature of the active and may be a product which typically comprises an aroma, for example a perfume, a washing product, a cleaning product, a cosmetic product, a personal care product, a hygiene article, a food, a food supplement or a fragrance dispenser. The product may alternatively be a pharmaceutical product, a crop protection product or an additive intended for use in building materials.

The present invention further provides for the use of compositions of the invention in the aforementioned products. Preference is given to the use of compositions of the invention in a product selected from perfumes, washing products, cleaning products, cosmetic products, personal care products, hygiene articles, foods, food supplements, fragrance dispensers and fragrances.

Compositions of the invention that comprise a fragrance as active may be used in the production of perfumed articles. The olfactory properties and also the physical properties and the non-toxicity of the compositions of the invention underline their particular suitability for the end uses mentioned.

The use of the compositions is found to be particularly advantageous in conjunction with top notes of compositions, by way of example in perfume compositions comprising dihydrorosan, rose oxides or other readily volatile fragrances, e.g. isoamyl acetate, prenyl acetate or methylheptenone. In this case, the release of the important, sought-after top notes is effectively delayed. The fragrance or aroma compositions are accordingly metered in at the suitable point in the requisite amount. In the mint compositions of L-menthol, DL-menthol, L-menthone and L-menthyl acetate described, aside from the aroma effect, a cooling effect is additionally applied in a targeted manner, e.g. in chewing gums, confectionery, cosmetic products, and industrial applications such as in textiles or superabsorbents. A further advantage lies in the high material compatibility of the compositions of the invention even with reactive or comparatively unstable components such as aldehydes, esters, pyrans/ethers, which may exhibit secondary reactions on the surfaces.

The positive properties contribute to use of the compositions of the invention with particular preference in perfume products, personal care products, hygiene articles, textile detergents and in cleaning products for solid surfaces.

The perfumed article is selected, for example, from perfume products, personal care products, hygiene articles, textile detergents and cleaning products for solid surfaces.

Preferred perfumed articles of the invention are also selected from:
perfume products selected from perfume extracts, eau de parfums, eau de toilettes, eau de colognes, eau de solide, extrait partum, air fresheners in liquid form, gel form or a form applied to a solid carrier, aerosol sprays, scented cleaners and scented oils;
personal care products selected from aftershaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, for example skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products, for example hairsprays, hair gels, setting hair lotions, hair conditioners, hair shampoo, permanent and semipermanent hair colorants, hair shaping compositions such as cold waves and hair smoothing compositions, hair tonics, hair creams and hair lotions, deodorants and antiperspirants, for example underarm sprays, roll-ons, deodorant sticks, deodorant creams, products of decorative cosmetics, for example eye shadows, nail varnishes, make-ups, lipsticks, mascara, toothpaste, dental floss;
hygiene articles selected from candles, lamp oils, joss sticks, propellants, rust removers, perfumed freshening wipes, armpit pads, baby diapers, sanitary towels, toilet paper, cosmetic wipes, pocket tissues, dishwasher deodorizer;
cleaning products for solid surfaces selected from perfumed acidic, alkaline and neutral cleaning products, for example floor cleaners, window cleaners, dishwashing detergents, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners;
textile detergents selected from liquid detergents, powder detergents, laundry pretreatments such as bleaches, soaking agents and stain removers, fabric softeners, washing soaps, washing tablets.

In a further aspect, the compositions of the invention are suitable for use in surfactant-containing perfumed articles. This is because there is frequently a search—especially for the perfuming of surfactant-containing formulations, for example cleaning products (in particular dishwashing compositions and all-purpose cleaners)—for odorants and/or odorant compositions with a rose top note and marked naturalness.

In a further aspect, the compositions of the invention can be used as products for providing (a) hair or (b) textile fibers with a rosy odor note.

The compositions of the invention are therefore of particularly good suitability for use in surfactant-containing perfumed articles.

It is preferred if the perfumed article is one of the following:
an acidic, alkaline or neutral cleaning product selected in particular from the group consisting of all-purpose cleaners, floor cleaners, window cleaners, dishwashing detergents, bath and sanitary cleaners, scouring milk, solid and liquid toilet cleaners, powder and foam carpet cleaners, liquid detergents, powder detergents, laundry pretreatments such as bleaches, soaking agents and stain removers, fabric softeners, washing soaps, washing tablets, disinfectants, surface disinfectants,
an air freshener in liquid form, gel-like form or a form applied to a solid carrier or as an aerosol spray,
a wax or a polish selected in particular from the group consisting of furniture polishes, floor waxes and shoe creams, or
a personal care product selected in particular from the group consisting of shower gels and shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, permanent and semi-permanent hair colorants, hair shaping compositions such as cold waves and hair smoothing compositions, hair tonics, hair creams and hair lotions, deodorants and antiperspirants such as e.g. underarm sprays, roll-ons, deodorant sticks, deodorant creams, products of decorative cosmetics.

The customary ingredients with which odorants used in accordance with the invention, or odorant compositions of the invention, may be combined are common knowledge and are described for example in WO2016/050836, the teaching of which is hereby expressly incorporated by reference.

Preference is likewise given to the use of compositions of the invention for controlled release of actives such as crop protecting agents and pharmaceutical agents.

### FIGURES

FIG. 1a shows a scanning electron micrograph of the microparticles from production example 1 before thermal treatment.

FIG. 1b shows a scanning electron micrograph of the microparticles from production example 1 after thermal treatment at 250° C.

FIG. 1c shows a scanning electron micrograph of the microparticles from example 1 after thermal treatment at 300° C.

FIG. 2 shows a scanning electron micrograph of the microparticles from production example 2 before thermal treatment.

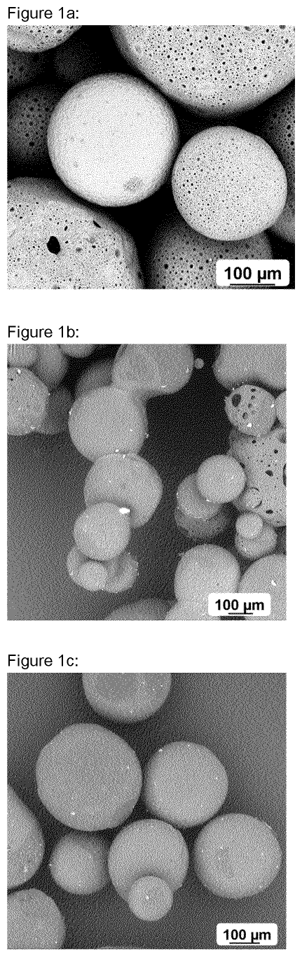

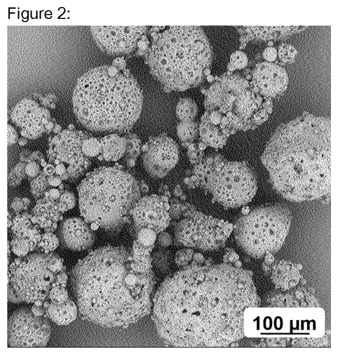

### EXAMPLES

Materials

Unless stated otherwise, the following materials and components were used:
Polybutylene sebacate terephthalate (PBSeT): Ecoflex™ FS Blend A1300, melting point in the range of 100-140° C., glass transition temperature of −33° C. (BASF SE), VST/A50=91° C.;
Amorphous polylactic acid (PLA-a): glass transition temperature of 55-60° C., VST/A50=56° C.;
Crystalline polylactic acid (PLA-a): melting point of 150° C. (BASF SE), VST/A50=56° C.;
Polyvinylalkohol: degree of hydrolysis of 88 mol %, a viscosity of a 4% by weight aqueous solution at 20° C. of 25 mPa*s and proportion of carboxyl groups of 3 mol %;
aroma chemical composition: mint aroma consisting of 2.3 wt.-% L-isopulegol, 3.1 wt.-% L-menthyl acetate, 36.4 wt.-% L-methone and 58.2 wt. % L-menthol characterized by the following evaporation rate at 25° C. and 1 bar:

| Time [hours] | Aroma chemical mixture Δ M [%]* |
|---|---|
| 0 | 0 |
| 3 | 15 |
| 10 | 45 |
| 19 | 87 |
| 33 | 99.1 |

*Decrease in mass of the aroma chemical mixture in % by weight normalized to the starting value

Methods

Determining the average particle diameter in aqueous suspension/emulsion by light scattering (laser diffraction).

The particle diameter of the w/o/w emulsion or the particle suspension is determined with a Malvern Mastersizer 2000 from Malvern Instruments, England, Hydro 2000S sample dispersion unit, by a standard test method documented in the literature. The value D[4,3] is the volume-weighted average.

Determining the average particle diameter of the solid:

The particle size distribution of the microparticles are determined as powder with a Malvern Mastersizer 2000 from Malvern Instruments, England, including Scirocco 2000 powder feed unit, by a standard test method documented in the literature. The value D[4,3] is the volume-weighted average.

Determining melting points: The melting temperature was determined by means of dynamic differential calorimetry (DSC) to DIN EN ISO 11357-3:2018-07 in open crucibles applying a heating rate of 10 K/min.

Determining glass transition temperature: The glass transition temperature was determined by means of dynamic differential calorimetry (DSC) to DIN EN ISO 11357-1:2017-02 in open crucibles applying a heating rate of 10 K/min.

Determining VST/A50: The Vicat softening Temperature was determined in accordance with the protocol of DIN EN ISO 306:2014-03 with a heating rate of 50° C./h and a force of 10 N.

Scanning electron microscopy: Close-up images were taken from a probe of the microparticles these were retrospectively automatically measured using the ProSuite (FibreMetric) software from Phenom.

Determining the Pore Diameter:

The pore diameters were determined by means of scanning electron microscopy as described above. The pores of a selected region of a particle were identified using the difference in contrast and the surfaces thereof were automatically measured. The diameter for each surface was calculated with the assumption that the surfaces were circular. (Sample size 100 pores).

In the context of the evaluation, only those pores whose pore diameter was at least 20 nm were taken into consideration. Depending on the particle size, the images were recorded, for larger particles with 1600- to 2400-times magnification, and for smaller particles with up to 8000-times magnification.

In order to determine the size of at least 10 pores, only those microparticles, whose particle diameter does not deviate from the mean particle diameter of the composition of microparticles by more than 20% were taken into consideration.

The following assumptions were made for evaluation of the number of pores based on the total surface area of the microparticle: Since these are spherical particles, the image only shows half the surface of the particle. If the image of a microparticle shows at least 5 pores whose diameter is at least 20 nm and whose diameter is in the range from 1/5000 to 1/5 of the mean particle diameter, then the total surface comprises at least 10 pores.

The evaluation was carried out according to the following procedure:
1. The mean particle diameter D[4,3] of the microparticles was already determined in the microparticle dispersion, using light scattering. The upper and lower limits of the particle diameter of the microparticles which are taken into consideration for determining the pores (±20%) can be calculated from this.
2. The microparticle dispersion was dried.
3. From a sample, in each case 20 images showing multiple microparticles were taken by means of scanning electron microscopy.
4. 20 microparticles were selected whose particle diameter is in the range ±20% of the mean particle diameter of the microparticles. The particle diameter thereof was thus measured with the ProSuite (FibreMetric) software from Phenom.
5. The pores of each of these 20 microparticles were measured. For this purpose, the surface areas of the visible pores were measured automatically and the diameter thereof was calculated.
6. The individual values of the pore diameters were checked as to whether their diameter met the condition of being in the range from 1/5000 to 1/5 of the mean particle diameter and being at least 20 nm.
7. The number of pores meeting this condition was determined and multiplied by two.
8. It was verified whether at least 16 microparticles had on average at least 10 pores.

### Production Example 1: Production of Spherical Fillable Microparticles

Spherical fillable microparticles were produced analogously to example 8 of WO 2018/065481. The matrix-forming polymer used was a polymer blend of 70% by weight of polybutylene sebacate terephthalate and 30% by weight of amorphous polylactic acid (PLA-a) (PBSeT; Ecoflex™ FS Blend A1300 product from BASF SE). This blend has a Vicat softening temperature VST/A50 of about 58.5° C. The procedure was as follows:

Pore former solution: 0.54 kg of ammonium carbonate was dissolved in 53.5 kg of water (pore former).

Solution of the aliphatic-aromatic polyester: 15.1 kg of PBSeT and 6.5 kg of PLA-a were stirred into 270.0 kg of dichloromethane and dissolved at 25° C. while stirring.

The w/o emulsion was produced by emulsifying the pore former solution in the solution of the aliphatic-aromatic polyester at 170 rpm with a twin-level cross-beam stirrer for 15 minutes.

The w/o emulsion thus obtained was transferred into 423 kg of a 0.8% b.w. solution of polyvinyl alcohol in water and likewise emulsified with shear and energy input (one minute at 120 rpm with an impeller stirrer).

Stirring of the w/o/w emulsion thus created was then continued with an impeller stirrer at 120 rpm, while reducing the pressure to 800 mbar and gradually increasing the jacket temperature to 40° C. and keeping it at this temperature for 4 hours. Thereafter, the microparticle suspension was cooled to room temperature, filtered and dried at 37° C.

The mean particle diameter D[v, 0.5] determined from the aqueous suspension was 270 μm. Bulk density was effected to DIN EN ISO 60:1999 and was 0.15 g/cm³. The pore size was 5.6 μm and was determined by means of mercury porosimetry. Visual evaluation was carried out as described above and showed than each of the microparticles within the size range of 210 to 325 μm had more than 10 pores at their surface, wherein the pores had a diameter within the range of 0.1 to 50 μm.

### Production Example 2: Production of Spherical Fillable Microparticles

The microparticles were prepared in accordance to the protocol of production example 1, where amorphous polylactic acid (PLA-a) was completely replaced by the same amount of crystalline polylactic acid (PLA-c). This blend has a Vicat softening temperature VST/A50 of about 57.2° C.

The mean particle diameter D[v, 0.5] determined from the aqueous suspension of the microparticles was 160 μm. Bulk density was effected to DIN EN ISO 60:1999 and was 0.15. g/cm³. The pore size was 5.0 μm and was determined by means of mercury porosimetry. Visual evaluation was carried out as described above and showed than each of the microparticles within the size range of 125 to 195 μm had more than 10 pores at their surface, wherein the pores had a diameter within the range of 0.1 to 30 μm.

### Production Example 3: Loading of the Microparticles with Aroma Chemical

The microparticles of production example 2 were loaded with the aroma chemical composition according to the following protocol.

4.0 g of the microparticles of production example 2 were placed in a plastic beaker equipped with a stirring bar. 8.0 g of the aroma chemical composition were dripped onto the microparticles at 23° C. while stirring for 10 min. to obtain the laden microparticles M P1. Any adherent liquid was removed by means of tissue and the obtained microparticles were weighed. The thus obtained microparticles contained about 60% by weight of the aroma chemical.

### Examples 1a, 1b, 2a and 2b

The heating zone was a vertically mounted steel pipe with a length of 3000 mm and an inside diameter of 55 mm, which was heated by means of an electric heating jacket over a length of 2000 mm. The temperature inside the pipe (internal temperature) was set to the desired temperature and controlled by a temperature sensor. The lower opening of the pipe was connected to a solids separator, which was a flask filled with cold water, the flask having an outlet just above the water surface, which was connected to a vacuum pump. An air flow was sucked downwards through the pipe by applying a slight vacuum to the solids separator. The flow rate was about 30 cm/s. By means of a funnel 5 g of the microparticles of production example 1 and 2, respectively were fed into the upper opening of the pipe. By this procedure a free flowing powder was obtained. The microparticles subjected to a heat treatment at an internal temperature of 250° C. showed only a few open pores (Example 1a and 2a respectively). At an internal temperature of 300° C. the pores were closed completely. No significant agglomeration was observed (Example 1 b and 2b).

### Example 3

The microparticles of production example 3 were subjected to a heat treatment according to the protocol of examples 1 and 2 at a temperature of 200° C. (example 3a) and 220° C. (sample 3b). At an internal temperature of 220° C. at least 80% of the pores of the particles were closed. Only slight agglomeration was observed.

The microparticle compositions from production example 3, example 3a and example 3b were stored together at 25° C. and a relative air humidity of 50% in a climate-controlled cabinet. The decrease in mass of the aroma chemical mixture was determined via the decrease in weight of the sample. The results are summarized in Table 1.

TABLE 1

| Time | Production example 3 | Example 3a | Example 3b |
|---|---|---|---|
| [hours] | Δ M [%]* | Δ M [%]* | Δ M [%]* |
| 0 | 0 | 0 | 0 |
| 10 | 86.7 | 81.2 | 70.8 |
| 33 | 99.9 | 83.7 | 85.7 |

*weight loss, based on the amount of perfume contained in the particles

1.-16. (canceled)

17. A process for producing microparticles laden with at least one active, wherein the microparticles are formed from a thermoplastic organic, polymeric material and in the unladen state, in their interior, have at least one cavity connected via pores to the surface of the microparticles, wherein
(a) a composition of unladen microparticles are impregnated with a liquid comprising the active, whereby laden microparticles are obtained, which contain in the interior cavity the liquid, and
(b) subjecting the laden microparticles to thermal treatment by passing a stream of free flowing laden microparticles in a carrier gas through a heated zone at a temperature of at least 20 K above the softening temperature of the thermoplastic organic polymeric material, where the average residence time of the laden microparticles in the heated zone is not more than 60 s.

18. The process according to claim 17, where the temperature in the heated zone is in the range from 130 to 350° C.

19. The process according to claim 17, where the softening temperature of the thermoplastic organic polymeric material is in the range from 50 to 160° C.

20. The process according to claim 17, where the heating zone has a straight tubular geometry.

21. The process according to claim 17, where the mass density of the stream of free flowing laden microparticles passed through the heated zone is in the range from 5 to 500 g/m³.

22. The process according to claim 17, where the stream of free flowing laden microparticles is quenched immediately after leaving the heated zone.

23. The process according to claim 17, wherein the thermoplastic organic, polymeric material comprises at least one polymer having a glass transition temperature or melting point in the range from 45 to 140° C.

24. The process according to claim 17, wherein the thermoplastic organic, polymeric material has a solubility in dichloromethane of at least 50 g/L at 25° C.

25. The process according to claim 17, wherein the thermoplastic polymeric material comprises at least one aliphatic-aromatic polyester or a combination of at least one aliphatic-aromatic polyester with at least on thermoplastic polymer which is not an aliphatic-aromatic polyester.

26. The process according to claim 25, wherein the thermoplastic organic, polymeric material, besides the aliphatic-aromatic polyester, additionally comprises at least one further polymer which is different from aliphatic-aromatic polyesters and which is in particular selected from the group consisting of aliphatic polyesters, polyanhydrides, polyesteramides, modified polysaccharides and proteins and mixtures thereof.

27. The process according to claim 17, wherein the active is liquid at 22° C. and 1013 mbar or has a melting point below 100° C.

28. The process according to claim 27, wherein the active is an aroma chemical which is liquid at 22° C. and 1013 mbar, or a mixture of aroma chemicals which is liquid at 22° C. and 1013 mbar.

29. A composition of microparticles filled with at least one active, obtainable by a process according to claim 17.

30. A product comprising a composition according to claim 29 in a proportion by weight of 0.01% to 80% by weight based on the total weight of the product.

31. The use of the composition according to claim 29 in a product selected from perfumes, washing and cleaning products, cosmetic products, personal care products, hygiene articles, foods, food supplements, fragrance dispensers and fragrances.

32. The use of the composition according to claim 29 for controlled release of actives.
